(19) United States
(12) Patent Application Publication
(10) Pub. No.: US 2022/0132834 A1
(43) Pub. Date: May 5, 2022

(54) METHODS, APPARATUSES, AND SYSTEMS FOR IMPROVING MICROBIAL PRESERVATION YIELD THROUGH RESCUE AND SERIAL PASSAGE OF PRESERVED CELLS

(71) Applicant: Native Microbials, Inc., San Diego, CA (US)

(72) Inventors: Sean GILMORE, San Diego, CA (US); Corey DODGE, Encinitas, CA (US)

(21) Appl. No.: 17/434,529

(22) PCT Filed: Feb. 28, 2020
(86) PCT No.: PCT/US2020/020311

§ 371 Date: Aug. 27, 2021

Related U.S. Application Data
(60) Provisional application No. 62/812,232, filed on Feb. 28, 2019.

(51) Int. Cl. A01N 1/02; C12N 1/20; C12N 1/14

(52) U.S. Cl. CPC A01N 1/0221; A01N 1/0284; C12N 1/14; C12N 1/20; A01N 1/0289

(57) ABSTRACT

The present disclosure provides methods of improving microbe viability after preservation comprising subjecting a population of target microbial cells to one or more preservation challenges and preparing a product using the population of preserved viability-enhanced microbial cells produced from said methods. The present disclosure further provides products comprising preserved viability-enhanced microbial cells produced by the methods described herein.

### CROSS-REFERENCE TO RELATED APPLICATIONS

This application claims priority to U.S. Provisional Application No. 62/812,232, filed on Feb. 28, 2019, the content of which is incorporated by reference in its entirety.

### DESCRIPTION OF THE TEXT FILE SUBMITTED ELECTRONICALLY

The sequence listing associated with this application is provided in text format in lieu of a paper copy, and is hereby incorporated by reference into the specification. The name of the text file containing the sequence listing is ASBI-017_01WO_ST25.txt. The text file is 8 kb, was created on Feb. 28, 2020, and is being submitted electronically via EFS-Web.

### BACKGROUND

Microorganisms coexist in nature as communities and engage in a variety of interactions, resulting in both collaboration and competition between individual community members. Advances in microbial ecology have revealed high levels of species diversity and complexity in most communities. Microorganisms are ubiquitous in the environment, inhabiting a wide array of ecosystems within the biosphere. Individual microorganisms and their respective communities play unique roles in environments such as marine sites (both deep sea and marine surfaces), soil, and animal tissues, including human tissue.

### SUMMARY

In some embodiments, the present disclosure provides a method of improving microbe viability after preservation comprising: subjecting a population of target microbial cells to a first preservation challenge to provide a population of challenged microbial cells; harvesting viable challenged microbial cells from the population of challenged microbial cells; preserving the viable challenged microbial cells to provide a population of preserved viability-enhanced microbial cells; and preparing a product using the population of preserved viability-enhanced microbial cells.

In some embodiments, the first preservation challenge includes one of freeze drying, lyophilization, cryopreservation, preservation by evaporation, preservation by foam formation, vitrification, stabilization by glass formation, preservation by vaporization, spray drying, adsorptive drying, extrusion, or fluid bed drying. In some embodiments, preserving the viable challenged cells includes freeze drying, lyophilization, cryopreservation, preservation by evaporation, preservation by foam formation, vitrification, stabilization by glass formation, preservation by vaporization, spray drying, adsorptive drying, extrusion drying, or fluid bed drying. In some embodiments, the population of challenged cells is subjected to at least one additional preservation challenge

In some embodiments, the present disclosure provides a method for microbe viability enhancement and preservation, the method comprising: subjecting a population of target microbial cells to a first preservation challenge to provide a first population of challenged microbial cells; harvesting viable challenged microbial cells from the first population of challenged microbial cells to provide a first population of viable challenged microbial cells; subjecting the first population of viable challenged microbial cells to a second preservation challenge to provide a second population of challenged microbial cells; harvesting viable challenged microbial cells from the second population of challenged microbial cells to provide a second population of viable challenged microbial cells; preserving the second population of viable challenged microbial cells to provide a population of preserved viability-enhanced microbial cells; and preparing a product using the population of preserved viability-enhanced microbial cells.

In some embodiments, the first preservation challenge and the second preservation challenge are of the same challenge type. In some embodiments, the first preservation challenge and the second preservation challenge are of different challenge types. In some embodiments, the first preservation challenge and the second preservation challenge are selected from a combination described in Table 1. In some embodiments, the second population of challenged cells is subjected to at least one additional preservation challenge. In some embodiments, preserving the second viable challenged cell population includes freeze drying, lyophilization, cryopreservation, preservation by evaporation, preservation by foam formation, vitrification, stabilization by glass formation, preservation by vaporization, spray drying, adsorptive drying, extrusion drying, or fluid bed drying.

In some embodiments, the population of target microbial cells comprises a Clostridium spp. bacterium, a Succinivibrio spp. bacterium, a Butyrivibio spp. bacterium, a Bacillus spp. bacterium, a Lactobacillus spp. bacterium, a Prevotella spp. bacterium, a Synrophococcus spp. bacterium, or a Ruminococcus spp. bacterium. In some embodiments, the population of target microbial cells comprises a Caecoryces spp. fungus, a Pichia spp. fungus, an Orpinomyces spp. fungus, or a Piromyces spp. fungus. In some embodiments, the population of target microbial cells comprises a species of the Lachnospiraceae family.

In some embodiments, the Clostridium spp. comprises a 16S rRNA sequence comprising at least 97% sequence identity to SEQ ID NO: 1, SEQ ID NO: 3, SEQ ID NO: 5, or SEQ ID NO: 6; the Succinivibrio spp. comprises a 16S rRNA sequence comprising at least 97% sequence identity to SEQ ID NO: 11; the Pichia spp. comprises an ITS sequence comprising at least 97% sequence identity to SEQ ID NO: 2; the Bacillus spp. comprises a 16S rRNA sequence comprising at least 97% sequence identity to SEQ ID NO: 4; the Lactobacillus spp. comprises a 16S rRNA sequence comprising at least 97% sequence identity to SEQ ID NO: 7, SEQ ID NO: 8, or SEQ ID NO: 9; the Prevotella spp. comprises a 16S rRNA sequence comprising at least 97% sequence identity to SEQ ID NO: 10; or the species of the Lachnospiraceae family comprises a 16S rRNA sequence comprising at least 97% sequence identity to SEQ ID NO: 12.

In some embodiments, the population of target microbial cells comprises a Ruminococcus bovis bacterium, a Succinivibrio dextrinosolvens bacterium, or a Caecomyces spp. fungus. In some embodiments, the population of target microbial cells comprises a Clostridium butyricum bacterium, a Pichia kudriazevii fungus, a Butyrivibio fibrosolvens bacterium, a Ruminococcus bovis bacterium, or a Succinivibrio dextrinosolvens bacterium.

In some embodiments, the present disclosure provides a product prepared by the methods described herein, comprising a population of preserved viability-enhanced microbial cells. In some embodiments, the population of preserved viability-enhanced microbial cells comprises a Clostridium spp. bacterium, a Succinivibrio spp. bacterium, a Caecomyces spp. fungus, a Pichia spp. fungus, a Butyrivibio spp. bacterium, an Orpinomyces spp. fungus, a Piromyces spp. fungus, a Bacillus spp. bacterium, a Lactobacillus spp. bacterium, a Prevotella spp. bacterium, a Syntrophococcus spp. bacterium, a Ruminococcus spp bacterium, or a species of the Lachnospiraceae family. In some embodiments, the Clostridium spp. comprises a 16S rRNA sequence comprising at least 97% sequence identity to SEQ ID NO: 1, SEQ ID NO: 3, SEQ ID NO: 5, or SEQ ID NO: 6; the Succinivibrio spp. comprises a 16S rRNA sequence comprising at least 97% sequence identity to SEQ ID NO: 11; the Pichia spp. comprises an ITS sequence comprising at least 97% sequence identity to SEQ ID NO: 2; the Bacillus spp. comprises a 16S rRNA sequence comprising at least 97% sequence identity to SEQ ID NO: 4; the Lactobacillus spp. comprises a 16S rRNA sequence comprising at least 97% sequence identity to SEQ ID NO: 7, SEQ ID NO: 8, or SEQ ID NO: 9; the Prevotella spp. comprises a 16S rRNA sequence comprising at least 97% sequence identity to SEQ ID NO: 10; or the species of the Lachnospiraceae family comprises a 16S rRNA sequence comprising at least 97% sequence identity to SEQ ID NO: 12.

### BRIEF DESCRIPTION OF THE FIGURES

FIG. 1 provides a process flow diagram illustrating a method according to the disclosure.

FIG. 2 provides a flow of challenge/rescue viability enhancement according to an embodiment of the disclosure.

FIG. 3 provides example results from applying disclosed methods to two different microbes.

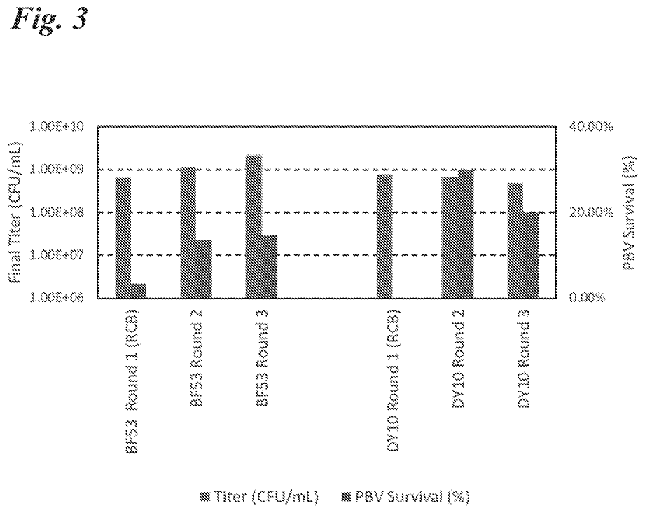

### DETAILED DESCRIPTION

### Overview

According to some embodiments of the disclosure, methods, apparatuses, and systems for challenge/rescue viability enhancement, including improving microbial stabilization/preservation yield via rescue and serial challenge/passage of cells. Such methods can be used for, by way of non-limiting example, in forming a synthetic ensemble, synthetic bioensemble, and/or live microbial product are disclosed. In some embodiments, such synthetic ensembles contain and/or comprise one or more stabilized and/or preserved microorganisms, for example, one or more microorganisms as disclosed in one or more of the following: U.S. Pat. App. Pub. Nos. 2018/0310592, 2018/0333443, and 2018/0223325 (each being herein expressly incorporated by reference for all purposes).

According to some embodiments of the disclosure, methods, apparatuses, and systems for challenge/rescue viability enhancement, including improving microbial stabilization/preservation yield via rescue and serial challenge/passage of cells. Such methods can be used for, by way of non-limiting example, in forming a synthetic ensemble, synthetic bioensemble, and/or live microbial product are disclosed. In some embodiments, such synthetic ensembles contain and/or comprise one or more stabilized and/or preserved microorganisms.

According to some embodiments, a target strain is identified. Then, once a target strain is identified, a first culture of the strain is grown, and cells are then harvested from the first culture. Once harvested, a pre-challenge baseline can be set/established and/or the initial viability tested. After harvesting, the cells are prepared for the challenge, for example, by combining with a preservation solution. An example preservation solution can include, by way of non-limiting example: an intracellular protectant (e.g., sugars, especially non-reducing sugars; sugar alcohols, such as sorbitol; and/or the like), a pH buffer (e.g., monosodium glutamate, monopotassium phosphate, dipotassium phosphate, and/or the like), a membrane protectant (e.g., polyvinyl-pyrrolidone K-15 and/or the like), as well as components to help with the preservation (e.g., where applicable, sucrose for glass formation, etc.) and quality control (e.g., a redox indicator such as resazurin for use with anaerobic microbes, etc.). Once the cells are prepared for the challenge, the first preservation challenge is performed. Examples of preservation/stabilization challenges can include, but are not limited to: freeze drying, lyophilization, cryopreservation, preservation by evaporation, preservation by foam formation, vitrification/stabilization by glass formation, preservation by vaporization, spray drying, adsorptive drying, extrusion, or fluid bed drying and/or the like. According to some embodiments, there can be multiple challenges prior to incorporation into and/or formation of the final product. In some embodiments, the challenge or challenges can be the same as the final preservation/stabilization, while in other embodiments, there may be more than one type of challenge used, each of which can be the same or different than the final preservation. For example, where PBV is the final stabilization/preservation step, the challenge or challenges can include a PBV challenge, and in some embodiments, can also include a cryopreservation challenge in addition to the PBV challenge and the final PBV process.

Once the first preservation challenge is performed, the challenged strain/preserved cells are prepared and grown in a rescue culture, and the cells from the rescue culture are harvested and viability is tested. The challenged strain can be prepared for and subjected to one or more additional challenges (which can be, as discussed above, the same or different from the previous challenge(s) and/or the final preservation/stabilization). Once the challenges have been completed, the surviving challenged cells are harvested from the rescue culture for preservation/stabilization, and the harvested challenged cells are preserved/stabilized to provide viability-enhanced cells. Then the viability-enhanced cells can be used for and/or incorporated into a final product, such as an ensemble, a live microbial feed additive, a live microbial feed supplement, and/or the like.

### Definitions

As used in this specification, the singular forms “a,” “an”, and “the” include plural referents unless the context clearly dictates otherwise. Thus, for example, the term “an organism type” is intended to mean a single organism type or multiple organism types. For another example, the term “an environmental parameter” can mean a single environmental parameter or multiple environmental parameters, such that the indefinite article “a” or “an” does not exclude the possibility that more than one of environmental parameter is present, unless the context clearly requires that there is one and only one environmental parameter.

Reference throughout this specification to “one embodiment”, “an embodiment”, “one aspect”, or “an aspect”, “one implementation”, or “an implementation” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure

As used herein, “carrier”, “acceptable carrier”, or “pharmaceutical carrier” refers to a diluent, adjuvant, excipient, or vehicle with which is used with or in the microbial ensemble. Such carriers can be sterile liquids, such as water and oils, including those of petroleum, animal, vegetable, or synthetic origin; such as peanut oil, soybean oil, mineral oil, sesame oil, and the like. Water or aqueous solution saline solutions and aqueous dextrose and glycerol solutions are preferably employed as carriers, in some embodiments as injectable solutions. Alternatively, the carrier can be a solid dosage form carrier, including but not limited to one or more of a binder (for compressed pills), a glidant, an encapsulating agent, a flavorant, and a colorant. The choice of carrier can be selected with regard to the intended route of administration and standard pharmaceutical practice. See Hardee and Baggo (1998. Development and Formulation of Veterinary Dosage Forms. 2nd Ed. CRC Press. 504 pg.); E.W. Martin (1970. Remington's Pharmaceutical Sciences. 17th Ed. Mack Pub. Co.); and Blaser et al. (US Publication US20110280840A1), each of which is herein expressly incorporated by reference in their entirety.

The terms “microorganism” and “microbe” are used interchangeably herein and refer to any microorganism that is of the domain Bacteria, Eukarya, or Archaea. Microorganism types include without limitation, bacteria (e.g., mycoplasma, coccus, bacillus, rickettsia, spirillum), fungi (e.g., filamentous fungi, yeast), nematodes, protozoans, archaea, algae, dinoflagellates, viruses (e.g., bacteriophages), viroids and/or a combination thereof. Organism strains are subtaxons of organism types, and can be for example, a species, sub-species, subtype, genetic variant, pathovar, or serovar of a particular microorganism.

As used herein, “spore” or “spores” refer to structures produced by bacteria and fungi that are adapted for survival and dispersal. Spores are generally characterized as dormant structures, however spores are capable of differentiation through the process of germination. Germination is the differentiation of spores into vegetative cells that are capable of metabolic activity, growth, and reproduction. The germination of a single spore results in a single fungal or bacterial vegetative cell. Fungal spores are units of asexual reproduction, and in some cases are necessary structures in fungal life cycles. Bacterial spores are structures for surviving conditions that may ordinarily be nonconductive to the survival or growth of vegetative cells. As used herein, “microbial composition” refers to a composition comprising one or more microbes of the present disclosure, wherein a microbial composition, in some embodiments, is administered to animals of the present disclosure.

As used herein, “individual isolates” should be taken to mean a composition, or culture, comprising a predominance of a single genera, species, or strain, of microorganism, following separation from one or more other microorganisms. The phrase should not be taken to indicate the extent to which the microorganism has been isolated or purified. However, “individual isolates” can comprise substantially only one genus, species, or strain, of microorganism.

As used herein, “microbiome” refers to the collection of microorganisms that inhabit the digestive tract or gastrointestinal tract of an animal (including the rumen if said animal is a ruminant) and the microorganisms' physical environment (i.e. the microbiome has a biotic and physical component). The microbiome is fluid and may be modulated by numerous naturally occurring and artificial conditions (e.g., change in diet, disease, antimicrobial agents, influx of additional microorganisms, etc.). The modulation of the microbiome of a rumen that can be achieved via administration of the compositions of the disclosure, can take the form of: (a) increasing or decreasing a particular Family, Genus, Species, or functional grouping of microbe (i.e. alteration of the biotic component of the rumen microbiome) and/or (b) increasing or decreasing volatile fatty acids in the rumen, increasing or decreasing rumen pH, increasing or decreasing any other physical parameter important for rumen health (i.e. alteration of the abiotic component of the rumen microbiome). As used herein, “probiotic” refers to a substantially pure microbe (i.e., a single isolate) or a mixture of desired microbes, and may also include any additional components that can be administered to a mammal for restoring microbiota. Probiotics or microbial inoculant compositions of the invention may be administered with an agent to allow the microbes to survive the environment of the gastrointestinal tract, i.e., to resist low pH and to grow in the gastrointestinal environment. In some embodiments, the present compositions (e.g., microbial compositions) are probiotics in some aspects.

The term “growth medium” as used herein, is any medium which is suitable to support growth of a microbe. By way of example, the media may be natural or artificial including gastrin supplemental agar, LB media, blood serum, and tissue culture gels. It should be appreciated that the media may be used alone or in combination with one or more other media. It may also be used with or without the addition of exogenous nutrients. The medium may be amended or enriched with additional compounds or components, for example, a component which may assist in the interaction and/or selection of specific groups of microorganisms. For example, antibiotics (such as penicillin) or sterilants (for example, quaternary ammonium salts and oxidizing agents) could be present and/or the physical conditions (such as salinity, nutrients (for example organic and inorganic minerals (such as phosphorus, nitrogenous salts, ammonia, potassium and micronutrients such as cobalt and magnesium), pH, and/or temperature) could be amended.

As used herein, “improved” should be taken broadly to encompass improvement of a characteristic of interest, as compared to a control group, or as compared to a known average quantity associated with the characteristic in question. For example, “improved” milk production associated with application of a beneficial microbe, or ensemble, of the disclosure can be demonstrated by comparing the milk produced by an ungulate treated by the microbes taught herein to the milk of an ungulate not treated. In the present disclosure, “improved” does not necessarily demand that the data be statistically significant (i.e. p<0.05); rather, any quantifiable difference demonstrating that one value (e.g. the average treatment value) is different from another (e.g. the average control value) can rise to the level of “improved.”

As used herein, “inhibiting and suppressing” and like terms should not be construed to require complete inhibition or suppression, although this may be desired in some embodiments. The term “marker” or “unique marker” as used herein is an indicator of unique microorganism type, microorganism strain, or activity of a microorganism strain. A marker can be measured in biological samples and includes without limitation, a nucleic acid-based marker such as a ribosomal RNA gene, a peptide- or protein-based marker, and/or a metabolite or other small molecule marker.

As used herein, the term “molecular marker” or “genetic marker” refers to an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Markers further include polynucleotide sequences encoding 16S or 18S rRNA, and internal transcribed spacer (ITS) sequences, which are sequences found between small-subunit and large-subunit rRNA genes that have proven to be especially useful in elucidating relationships or distinctions among when compared against one another. Mapping of molecular markers in the vicinity of an allele is a procedure which can be performed by the average person skilled in molecular-biological techniques.

As used herein, the term “trait” refers to a characteristic or phenotype. For example, in the context of some embodiments of the present disclosure, quantity of milk fat produced relates to the amount of triglycerides, triacylglycerides, diacylglycerides, monoacylglycerides, phospholipids, cholesterol, glycolipids, and fatty acids present in milk. Desirable traits may also include other milk characteristics, including but not limited to: predominance of short chain fatty acids, medium chain fatty acids, and long chain fatty acids; quantity of carbohydrates such as lactose, glucose, galactose, and other oligosaccharides; quantity of proteins such as caseins and whey; quantity of vitamins, minerals, milk yield/volume; reductions in methane emissions or manure; improved efficiency of nitrogen utilization; improved dry matter intake; improved feed efficiency and digestibility; increased degradation of cellulose, lignin, and hemicellulose; increased rumen concentrations of fatty acids such as acetic acid, propionic acid, and butyric acid; etc.

A trait may be inherited in a dominant or recessive manner, or in a partial or incomplete-dominant manner. A trait may be monogenic (i.e. determined by a single locus) or polygenic (i.e. determined by more than one locus) or may also result from the interaction of one or more genes with the environment. In the context of this disclosure, traits may also result from the interaction of one or more mammalian genes and one or more microorganism genes.

As used herein, the term “homozygous” means a genetic condition existing when two identical alleles reside at a specific locus, but are positioned individually on corresponding pairs of homologous chromosomes in the cell of a diploid organism. Conversely, as used herein, the term “heterozygous” means a genetic condition existing when two different alleles reside at a specific locus, but are positioned individually on corresponding pairs of homologous chromosomes in the cell of a diploid organism.

As used herein, the term “phenotype” refers to the observable characteristics of an individual cell, cell culture, organism (e.g., a ruminant), or group of organisms which results from the interaction between that individual's genetic makeup (i.e., genotype) and the environment.

As used herein, the term “chimeric” or “recombinant” when describing a nucleic acid sequence or a protein sequence refers to a nucleic acid, or a protein sequence, that links at least two heterologous polynucleotides, or two heterologous polypeptides, into a single macromolecule, or that re-arranges one or more elements of at least one natural nucleic acid or protein sequence. For example, the term “recombinant” can refer to an artificial combination of two otherwise separated segments of sequence, e.g., by chemical synthesis or by the manipulation of isolated segments of nucleic acids by genetic engineering techniques.

As used herein, a “synthetic nucleotide sequence” or “synthetic polynucleotide sequence” is a nucleotide sequence that is not known to occur in nature or that is not naturally occurring. Generally, such a synthetic nucleotide sequence will comprise at least one nucleotide difference when compared to any other naturally occurring nucleotide sequence.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue” or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar, or strain. For purposes of this disclosure homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

As used herein, the term “nucleotide change” refers to, e.g., nucleotide substitution, deletion, and/or insertion, as is well understood in the art. For example, mutations contain alterations that produce silent substitutions, additions, or deletions, but do not alter the properties or activities of the encoded protein or how the proteins are made.

As used herein, the term “protein modification” refers to, e.g., amino acid substitution, amino acid modification, deletion, and/or insertion, as is well understood in the art.

As used herein, the term “at least a portion” or “fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

Variant polynucleotides also encompass sequences derived from a mutagenic and recombinogenic procedure such as DNA shuffling. Strategies for such DNA shuffling are known in the art. See, for example, Stemmer (1994) PNAS 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) PNAS 94:4504-4509; Crameri et at (1998) Nature 391:288-291; and U.S. Pat. Nos. 5,605,793 and 5,837,458. For PCR amplifications of the polynucleotides disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (1989) Molecular Cloning: A Laboratory Manual (2nd ed., Cold Spring Harbor Laboratory Press, Plainview, N.Y.). See also Innis et at, eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

As used herein, the term “MIC” means maximal information coefficient. MIC is a type of nonparametric network analysis that identifies a score (MIC score) between active microbial strains of the present disclosure and at least one measured metadata (e.g., milk fat). Further, U.S. application Ser. No. 15/217,575, filed on Jul. 22, 2016 (issued as U.S. Pat. No. 9,540,676 on Jan. 10, 2017) is hereby incorporated by reference in its entirety.

As used herein “shelf-stable” refers to a functional attribute and new utility acquired by the microbes formulated according to the disclosure, which enable said microbes to exist in a useful/active state outside of their natural environment (i.e. a markedly different characteristic). Thus, shelf-stable is a functional attribute created by the formulations/compositions of the disclosure and denoting that the microbe formulated into a shelf-stable composition can exist outside the natural environment and under ambient conditions for a period of time that can be determined depending upon the particular formulation utilized, but in general means that the microbes can be formulated to exist in a composition that is stable under ambient conditions for at least a few days and generally at least one week.

### Serial Preservation Methods

In some embodiments, the present disclosure provides methods of improving microbe viability after preservation by subjecting the microbial cultures to serial preservation challenges and preparing a product from the population of viable, preservation challenged microbes present in culture at the conclusion of the preservation challenges. In some embodiments, the microbial cultures are subjected to at least one preservation challenge. In some embodiments, the microbial cultures are subjected to at least two, three, four, five, or more preservation challenges.

In some embodiments, the present disclosure provides a method of improving microbe viability after preservation comprising: (a) subjecting a population of target microbial cells to a first preservation challenge to provide a population of challenged microbial cells; (b) harvesting viable challenged microbial cells from the population of challenged microbial cells; (c) preserving the viable challenged microbial cells to provide a population of preserved viability-enhanced microbial cells; and (d) preparing a product using the population of preserved viability-enhanced microbial cells.

In some embodiments, the present disclosure provides a method for microbe viability enhancement and preservation, the method comprising: (a) subjecting a population of target microbial cells to a first preservation challenge to provide a first population of challenged microbial cells; (b) harvesting viable challenged microbial cells from the first population of challenged microbial cells to provide a first population of viable challenged microbial cells; (c) subjecting the first population of viable challenged microbial cells to a second preservation challenge to provide a second population of challenged microbial cells; (d) harvesting viable challenged microbial cells from the second population of challenged microbial cells to provide a second population of viable challenged microbial cells; (e) preserving the second population of viable challenged microbial cells to provide a population of preserved viability-enhanced microbial cells; and (f) preparing a product using the population of preserved viability-enhanced microbial cells.

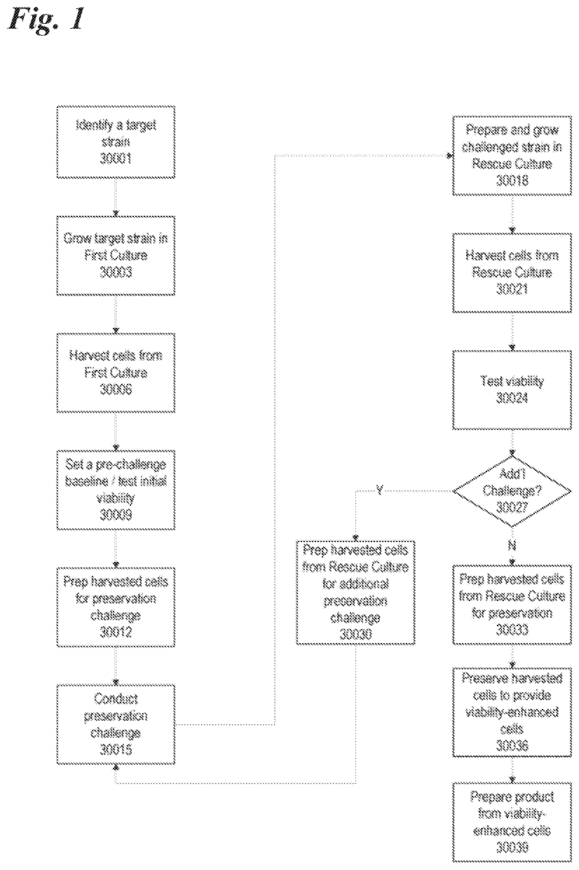

According to some embodiments, and as illustrated by the flow diagram in FIG. 1, a target strain is identified 30001. Identifying the target strain can include one or more of the discovery methods as detailed in U.S. Pat. No. 9,938,558, the entirety of which is herein expressly incorporated by reference for all purposes. For example, in one aspect of the disclosure, a method for identifying one or more active microorganisms from a plurality of samples is disclosed, and includes: determining the absolute cell count of one or more active microorganism strains in a sample, and analyzing microorganisms with at least one metadata, wherein the one or more active microorganism strains is present in a microbial community in the sample. The one or more microorganism strains can be a subtaxon of a microorganism type.

Then, once a target strain is identified 30001, a first culture of the strain is grown 30003. Cells are then harvested from the first culture 30006. Once harvested 30006, a pre-challenge baseline can be established and/or the initial viability tested 30009. Once harvested, the cells are prepared for the challenge 30012, for example, by combining with a preservation solution.

Once the cells are prepared for the challenge 30012, the first preservation challenge is performed 30015. Examples of preservation challenges include, but are not limited to: freeze drying (also known as lyophilization), preservation by vitrification (also known as preservation by glass formation), preservation by evaporation, preservation by foam formation (PFF), preservation by vaporization (PBV), cryopreservation, spray drying, adsorptive drying, extrusion, fluid bed drying, and/or the like.

According to some embodiments, there can be multiple challenges prior to incorporation into the final product. In some embodiments, the challenge or challenges can be the same as the final preservation, while in other embodiments, there may be more than one type of challenge used, each of which can be the same or different than the final preservation. For example, where PBV is the final stabilization/preservation step, the challenge or challenges can include a PBV challenge, and in some embodiments, can also include a cryopreservation challenge in addition to the PBV challenge and the final PBV process.

Once the first preservation challenge is performed 30015, the challenged microbial cells are prepared and grown in a rescue culture 30018, and the cells from the rescue culture are harvested 30021, the viability is tested 30024. The challenged strain can 30027 be prepared for additional preservation challenges 30030 and subjected to one or more additional preservation challenges 30015 (which can be, as discussed above, the same or different from the previous challenge(s) and/or the final preservation).

Once the challenges have been completed 30027, the surviving challenged cells are harvested from the rescue culture for preservation 30033, and the harvested challenged cells are preserved 30036 to provide viability-enhanced cells 30036. Then the viability-enhanced cells can be incorporated into a final product, such as an ensemble, a live microbial feed additive, a live microbial supplement, and/or the like.

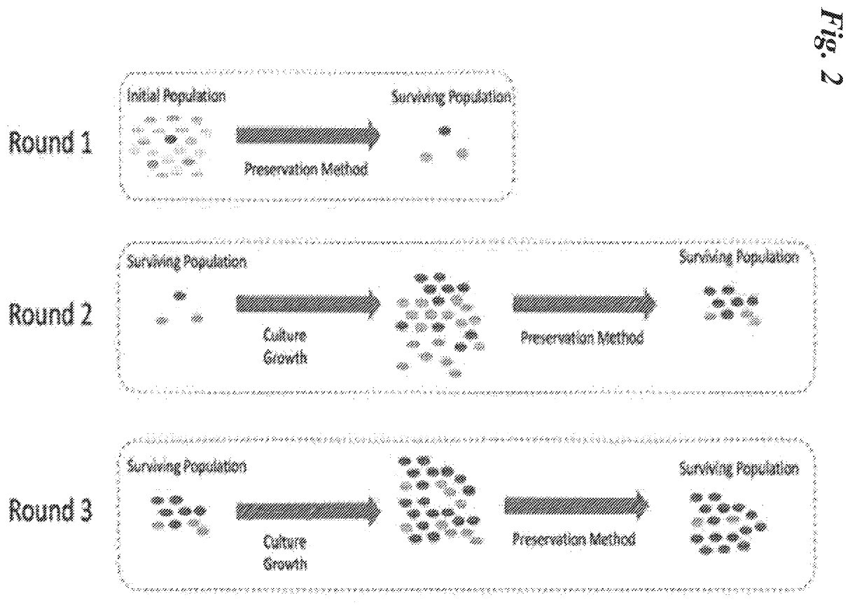

FIG. 2 provides an additional schematic of the serial preservation challenge methods described herein. Additionally, in some embodiments, genetic analyses of a strain are performed to compare microbial populations subjected to preservation challenges and those not subjected to preservation challenges.

In some embodiments, the methods provided herein comprising serial preservation of microbial cultures result in an increase in microbial viability of at least 5%. In other words, the viability of the population of microbes present at the conclusion of the serial preservation challenges is increased by at least 5% compared to the viability of the population of microbes that were present prior to any preservation challenges. In some embodiments, the methods provided herein comprising serial preservation of microbial cultures result in an increase in microbial viability between about 5% and about 30%, about 5% and about 25%, about 5% and about 20%, about 5% and about 15%, about 5% and about 10%, about 10% and about 30%, about 15% and about 30%, about 20% and about 30%, or about 25% and about 30%. In some embodiments, the methods provided herein comprising serial preservation of microbial cultures result in an increase in microbial viability between about 10% and about 30%, about 15% and about 30%, about 20% and about 30%, about 25% and about 30%, about 10% and about 25%, about 10% and about 20%, or about 10% and about 15%. In some embodiments, the methods provided herein comprising serial preservation of microbial cultures result in an increase in microbial viability of at least 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29/c, 30% or more.

### Preservation Challenges

In some embodiments, the present disclosure provides methods of improving microbe viability after preservation by subjecting the microbial cultures to serial preservation challenges, wherein microbes are subjected to one or more preservation challenges. In some embodiments, the microbes are subjected to two, three, four, five, or more preservation challenges before final preservation for storage and/or incorporation into a product.

In some embodiments, each of the preservation challenges are the same type of preservation challenge. For example, in some embodiments, the microbes are subjected to two, three, four, five, or more preservation challenges before final preservation for storage and/or incorporation into a product, wherein each of the preservation challenges are of the same type (e.g., are each freeze drying/lyophilization, are each preservation by vitrification/glass formation, are each preservation by evaporation, are each preservation by foam formation, are each preservation by vaporization, are each cryopreservation, are each spray drying, are each adsorptive drying, are each extrusion, or are each fluid bed drying).

In some embodiments, the preservation challenges are different types of preservation challenges. For example, in some embodiments, the microbes are subjected to a first and a second preservation challenge, wherein the first and the second preservation challenges are different challenges types. For example, in some embodiments, the first preservation challenge is a cryopreservation challenge and the second preservation challenge is a freeze-drying preservation challenge. Exemplary combinations of preservation challenge types are provided in Table 1 below.

TABLE 1
Preservation Challenge Combinations

| First Preservation Challenge | Second Preservation Challenge |
|---|---|
| Freeze drying | Freeze drying |
| Freeze drying | Vitrification |
| Freeze drying | Evaporation |
| Freeze drying | Foam formation |
| Freeze drying | Vaporization |
| Freeze drying | Cryopreservation |
| Freeze drying | Spray drying |
| Freeze drying | Adsorptive drying |
| Freeze drying | Extrusion |
| Freeze drying | Fluid bed drying |
| Vitrification | Freeze drying |
| Vitrification | Vitrification |
| Vitrification | Evaporation |
| Vitrification | Foam formation |
| Vitrification | Vaporization |
| Vitrification | Cryopreservation |
| Vitrification | Spray drying |
| Vitrification | Adsorptive drying |
| Vitrification | Extrusion |
| Evaporation | Freeze drying |
| Evaporation | Vitrification |
| Evaporation | Evaporation |
| Evaporation | Foam formation |
| Evaporation | Vaporization |
| Evaporation | Cryopreservation |
| Evaporation | Spray drying |
| Evaporation | Adsorptive drying |
| Evaporation | Extrusion |
| Foam formation | Freeze drying |
| Foam formation | Vitrification |
| Foam formation | Evaporation |
| Foam formation | Foam formation |
| Foam formation | Vaporization |
| Foam formation | Cryopreservation |
| Foam formation | Spray drying |
| Foam formation | Adsorptive drying |
| Foam formation | Extrusion |
| Vaporization | Freeze drying |
| Vaporization | Vitrification |
| Vaporization | Evaporation |
| Vaporization | Foam formation |
| Vaporization | Vaporization |
| Vaporization | Cryopreservation |
| Vaporization | Spray drying |
| Vaporization | Adsorptive drying |
| Vaporization | Extrusion |
| Cryopreservation | Freeze drying |
| Cryopreservation | Vitrification |
| Cryopreservation | Evaporation |
| Cryopreservation | Foam formation |
| Cryopreservation | Vaporization |
| Cryopreservation | Cryopreservation |
| Cryopreservation | Spray drying |
| Cryopreservation | Adsorptive drying |
| Cryopreservation | Extrusion |
| Spray drying | Freeze drying |
| Spray drying | Vitrification |
| Spray drying | Evaporation |
| Spray drying | Foam formation |
| Spray drying | Vaporization |
| Spray drying | Cryopreservation |
| Spray drying | Spray drying |
| Spray drying | Adsorptive drying |
| Spray drying | Extrusion |
| Adsorptive drying | Freeze drying |
| Adsorptive drying | Vitrification |
| Adsorptive drying | Evaporation |
| Adsorptive drying | Foam formation |
| Adsorptive drying | Vaporization |
| Adsorptive drying | Cryopreservation |
| Adsorptive drying | Spray drying |
| Adsorptive drying | Adsorptive drying |
| Adsorptive drying | Extrusion |
| Extrusion | Freeze drying |
| Extrusion | Vitrification |
| Extrusion | Evaporation |
| Extrusion | Foam formation |
| Extrusion | Vaporization |
| Extrusion | Cryopreservation |
| Extrusion | Spray drying |
| Extrusion | Adsorptive drying |
| Extrusion | Extrusion |

### Freeze-Drying (FD)/Lyophilization

In some embodiments, a population of target microbial cells is subjected to preservation by freeze-drying (also referred to as preservation by lyophilization). Freeze-drying, or lyophilization, has been known and applied to preserve various types of proteins, cells, viruses, and microorganisms. FD typically comprises primary drying and secondary drying. Freeze-drying can be used to produce stable bio-actives in industrial quantities. Freeze-drying can be damaging to cellular components, and can result in reduced viability, and conventionally freeze-dried products are typically only stable at or near 0° C., which can require that the bioactive material product be refrigerated from the time it is manufactured until the time it is utilized, requiring refrigeration during storage and transportation.

### A. Primary Freeze-Drying

The limitations of freeze-drying, as described above, result in part from a need to utilize low pressure (or high vacuum) during a freeze-drying process. A high vacuum is required because the temperature of the material during the primary freeze-drying should be below its collapse temperature, which is approximately equal to Tg′. At such low temperatures, the primary drying takes many hours (sometimes days) because the equilibrium pressure above ice at temperatures below −25° C. is less than 0.476 Torrs. Therefore, a new process must allow for shorter production times.

The low vacuum pressure used in freeze-drying methods limits the amount of water that can be removed from drying. Primary freeze-drying is performed by sublimation of ice from a frozen specimen at temperatures close to or below Tg′ that is a temperature at which a solution that remains not frozen between ice crystals becomes solid (vitrifies) during cooling. According to conventional beliefs, performing freeze-drying at such low temperatures is important for at least two reasons. The first reason for which freeze-drying at low temperatures (i.e., below Tg′) is important is to ensure that the cake remaining after ice removal by sublimation (primary drying) is “solid” and mechanically stable, i.e., that it does not collapse. Keeping the cake in a mechanically stable “solid” state after primary freeze-drying is important to ensure effective reconstitution of the freeze-dried material. Several methods were proposed to measure the Tg′ for a specific material. These methods rely on different interpretations of the features that can be seen in DSC (Differential Scanning Calorimeter) thermograms. The most reliable way to determine Tg′ is based on an evaluation of the temperature at which ice begins to melt and the concentration of water remaining unfrozen (Wg′) during slow cooling. The second reason typically advanced to support the importance of freeze-drying at low temperatures (i.e., below Tg′) is that the survival rate of bio-actives after freeze-drying is higher if the primary freeze-drying is performed at lower temperatures.

FD can be damaging for sensitive bio-actives. Strong FD-induced injury occurs during both freezing (formation of ice crystals) and the subsequent equilibration of the frozen specimens at intermediately low temperatures during ice sublimation. Well-known factors that cause cell damage during freezing include: freeze-induced dehydration, mechanical damage of cells during ice crystallization and recrystallization, phase transformation in cell membranes, increasing electrolyte concentration and others. Additionally, damages to frozen bio-actives can be caused by large pH change in the liquid phase that remains unfrozen between ice crystals. This abnormal pH change is associated with crystallization hydrolysis.

Crystallization hydrolysis occurs because ice crystals capture positive and negative ions differently. This creates a significant (about 107 V/m) electrical field inside ice crystals. Neutralization of this electrical field occurs due to electrolysis inside the ice crystals at a rate proportional to the constant of water molecule dissociation in ice. This neutralization results in a change of the pH of the liquid that remains between the ice crystals. The damaging effect of crystallization hydrolysis can be decreased by reducing the surface of ice that forms during freezing and by increasing the volume of the liquid phase that remains between the ice crystals. This remaining liquid also reduces the damaging effect of (i) the increasing electrolyte (or any other highly reactive molecules) concentration and (ii) the mechanical damage to cells between the ice crystals. The increase of the liquid between the ice crystals can be achieved by (i) increasing the initial concentration of protectants added before freezing, and (ii) by decreasing the amount of ice formed in the sample.

Avoiding freezing to temperatures equal to Tg′ or below (at which freeze-drying is typically performed) will allow to significantly reduce the amount of damage in the preserved biological. Therefore, a new method that allows a preservation of bio-actives without subjecting the bio-actives to temperatures near or below Tg′ will significantly improve the quality of the preserved material.

### B. Secondary Freeze-Drying

After the removal of ice by sublimation (primary drying) is complete, the sample may be described as a porous cake. Concentration of water in the sample at the end of primary drying is above the concentration of water that remains unfrozen in the glassy channels between ice crystals at a temperature below Tg′ (Wg′). Tg′ strongly depends on the composition of the solution, while for the majority of solutes Wg′ is about 20 wt %. At such high water concentrations, the glass transition temperature of the cake material is below the primary freeze-drying temperature, and/or significantly below −20° C. Secondary drying is performed to remove the remaining (about 20 wt %) water and increase the glass transition temperature in the cake material. As a practical matter, secondary drying cannot be performed at Tg′ or lower temperatures because diffusion of water from a material in a glass state is extremely slow. For this reason, secondary drying is performed by heating the cake to a drying temperature Td that is higher than the glass transition temperature Tg of the cake material at a given moment. If during the secondary drying step, Td is substantially higher than Tg, the cake will “collapse” and form a very viscous syrup, thereby making standard reconstitution impossible. Therefore, the collapse of the cake is highly undesirable.

The collapse phenomenon, which is kinetic by nature, has been extensively discussed in the literature. The rate of the collapse increases as the viscosity of the cake material decreases. To avoid or bring the collapse process to a negligible scale, Td is kept close to Tg during the secondary drying, thereby ensuring that the viscosity of the cake material is high and the rate of the collapse slow.

### Preservation by Vitrification (Glass Formation)

In some embodiments, a population of target microbial cells is subjected to preservation by vitrification. “Preservation by vitrification” is a transformation from a liquid into a highly immobile, noncrystalline, amorphous solid state, known as the “glass state.” Such a process may also be referred to as “preservation by glass formation”. A “glass state” is an amorphous solid state, which may be achieved by supercooling of a material that was initially in a liquid state. Diffusion in vitrified materials (e.g., “glass”) occurs at extremely low rates. Consequently, chemical and biological changes requiring the interaction of more than one moiety are practically completely inhibited. Glass typically appear as homogeneous, transparent, brittle solids, which can be ground or milled into a powder. Above a temperature known as the glass transition temperature (Tg), the viscosity drops rapidly and the material transforms from a glass state into what is known as a deformable “rubber state.” As the temperature increases, the material transitions into a liquid state. The optimal benefits of vitrification for long-term storage may be secured only under conditions where Tg is greater than the storage temperature.

Vitrification has been broadly used to preserve biological and highly reactive chemicals. The basic premise of vitrification is that all diffusion limited physical processes and chemical reactions, including the processes responsible for the degradation of biological materials, stop in the glass state. In general terms, glasses are thermodynamically unstable, amorphous materials that are mechanically stable at their very high viscosity (1012-1014 Pa/s.). A typical liquid has a flow rate of 10 m/s compared to 10⁻¹⁴ m/s in the glass state.

Bio-actives can be preserved at −196° C. Tg for pure water is about −145° C. If ice crystals form during cooling, the solution that remains unfrozen in the channels between the ice crystals will vitrify at Tg′, which is higher than Tg for pure water. Bio-actives that are rejected in the channels during ice growth will be stable at temperatures below Tg′. Bio-actives can be stabilized at temperatures substantially higher than −145° C. provided they are placed in concentrated preservation solutions with high Tg. For example, for a solution that contains 80% sucrose, Tg is about −40° C. A solution that contains 99% sucrose is characterized by Tg of about 52° C. The presence of water in a sample results in a strong plasticizing effect, which decreases Tg. The Tg is directly dependent on the amount of water present, and may, therefore, be modified by controlling the level of hydration—the less water, the higher the Tg. Therefore, the specimens (to be vitrified at an ambient temperature) must be strongly dehydrated by drying. However, drying can be damaging to bio-actives. Therefore, to stabilize bio-actives at a room temperature and still preserve their viability and functions, they need to be dried in the presence of a protective excipient (i.e., protectant) or a combination of excipients, which have a glass transition temperature Tg higher than the room temperature.

### Preservation by Evaporation

In some embodiments, a population of target microbial cells is subjected to preservation by evaporation. “Preservation by evaporation” refers to a process comprising the removal of water by evaporative drying.

In some embodiments, activity of bio-actives dried by evaporative drying of small drops is comparable to the activity of freeze-dried samples. For example, it has been shown that labile enzymes (luciferase and isocitric dehydrogenase) can be preserved by evaporative drying for more than a year at 50° C. without any detectable loss of activity during drying and subsequent storage at 50° C. Because dehydrated solutions containing protectors become viscous, it can take long periods of time to evaporate water even from small drops of a solution.

### Preservation by Foam Formation

In some embodiments, a population of target microbial cells is subjected to preservation by foam formation. During preservation by foam formation (PFF), the biological materials are first transformed into mechanically stable, dry foams by boiling them under vacuum at ambient temperatures above the freezing point (referred to as primary drying). Second the sample are subjected to stability drying at elevated temperature to increase the glass-transition temperature. Survival or activity yield after rehydration of preserved samples is achieved by proper selection of protectors (e.g., sugars) that are dissolved in the suspension before PFF and by proper selection of the vacuum and temperature protocols during PFF (See, Bronshtein, Victor. (2004). Bronshtein 2004 Preservation by Foam Formulation. PharmTech. Pharmaceutical Technology. 28. 86-92).

### Preservation by Vaporization

In some embodiments, a population of target microbial cells is subjected to preservation by vaporization. Preservation by Vaporization (PBV) is a preservation process that comprises primary drying and stability drying. Primary drying is performed by intensive vaporization (sublimation, boiling, and evaporation) of water at temperatures significantly higher (approximately 10° C. or more) than Tg′ from a partially frozen and at the same time overheated material (i.e., where the vacuum pressure is below the equilibrium pressure of water vapor).

During PBV, the boiling in the course of the primary drying does not produce a lot of splattering because the equilibrium pressure at subzero temperatures above the slush is low and ice crystals on the surface of the slush prevent or inhibit the splattering. Typically, a material (e.g., frozen solutions or suspensions) which has been subjected to PBV drying looks like a foam partly covered with a skim of a thin freeze-dried cake.

Unlike preservation by foam formation (PFF), preservation by vaporization (PBV) can be very effective for preserving bio-actives contained or incorporated within an alginate gel formulation and other gel formulations. A PBV process can be performed by drying frozen gel particles under a vacuum at small negative (on the Celsius scale) temperatures. For such hydrogel systems, vaporization comprises simultaneous sublimation of ice crystals, boiling of water inside unfrozen micro inclusions, and evaporation from the gel surface.

PBV can be different from freeze-drying because freeze-drying suggests the product processing temperature to be at or below Tg′ (which, typically, is below −25° C.) during primary drying and because freeze-drying suggests avoiding the “collapse” phenomenon during both primary and secondary drying. PBV comprises drying at temperatures substantially higher than T_{g}′, i.e., higher than −15° C., better higher than −10° C., and yet better higher than −5° C.

Additional details about PBV and other challenges can be found in U.S. Pat. App. Pub. No. 2008/0229609, the entirety of which is hereby expressly incorporated by reference herein for all purposes.

### Cryopreservation

In some embodiments, a population of target microbial cells is subjected to cryopreservation. Cryopreservation refers to the use of very low temperatures to preserve structurally intact living cells and tissues. The damaging effect of cryopreservation is mostly associated with freeze-induced dehydration, change in pH, increase in extracellular concentration of electrolytes, phase transformation in biological membranes and macromolecules at low temperatures, and other processes associated with ice crystallization. Potential cryodamage is a drawback in the methods that rely on freezing of bio-actives. This damage can be decreased by using cryoprotective excipients (protectants), e.g., glycerol, ethylene glycol, dimethyl sulfoxide (DMSO), sucrose and other sugars, amino acids, synthetic, and/or biological polymers, etc.

### Spray Drying

In some embodiments, a population of target microbial cells is subjected to preservation by spray drying. Spray drying refers to a method of producing a dry powder from a liquid or slurry by rapidly drying with a hot gas. Spray-drying generally comprises spraying, in a chamber, a suspension of microorganisms in a stream of hot air, the chamber comprising an inlet for heated air, an outlet for discharging air, and an outlet for recovering the powder of dried microorganisms. Exemplary temperatures, chamber volumes, and gases for use in spray drying methods can be found in U.S. Pat. No. 6,010,725.

### Adsorptive Drying

In some embodiments, a population of target microbial cells is subjected to preservation by adsorptive drying. Adsorptive drying refers to a method comprising the removal of water by diffusion into and adsorption onto porous materials such as aluminas, silica gels, molecular sieves, and other chemical drying agents.

### Extrusion

In some embodiments, a population of target microbial cells is subjected to preservation by extrusion. Extrusion refers to a method in which materials are forced through a die in order to shape them. In some embodiments, the target microbial cells are dispersed in a carrier or matrix in order to protect them from oxygen, heat, moisture, and the like.

### Fluid Bed Drying

In some embodiments, a population of target microbial cells is subjected to preservation by fluid bed drying. Fluid bed drying refers to a method in which particles are fluidized in a bed and dried. A fluidized bed is formed when a quantity of solid particulates are placed under conditions that cause a solid material to behave like a fluid. In a fluid bed drying system, inlet air provides significant air flow to support the weight of the particles.

### Stability Drying

In some embodiments, a population of target microbial cells is subjected to preservation by a drying method (e.g., freeze-drying, preservation by vitrification/glass formation, preservation by evaporation, preservation by foam formation, preservation by vaporization, spray drying, adsorptive drying, or fluid bed drying) and the drying preservation method further comprises stability drying. The stability drying is performed (1) to further increase the glass transition temperature of the dry material, (2) to make it mechanically stable at ambient temperatures without vacuum, and (3) to preserve the potency and efficacy of the biological during a long-term storage at ambient temperatures.

To increase Tg of the material to for example 37° C. and to thereby ensure stabilization at this temperature, the stability drying step should be performed at temperatures significantly higher than 37° C. over many hours to remove water from inside of already dried material.

The process of dehydration of biological specimens at elevated temperatures may be very damaging to the subject bio-actives if the temperature used for drying is higher than the applicable protein denaturation temperature. To protect the sample from the damage that can be caused by elevated temperatures, the stability dehydration process (i.e., stability drying) may need to be performed in steps. The first step (either in air or vacuum) should be performed at a starting temperature to ensure dehydration without a significant loss of a biological's viability and potency. After such first drying step, the process of dehydration may be continued in subsequent steps by drying at a gradually higher temperature during each subsequent step. Each step will allow simultaneous increases in the extent of the achievable dehydration and the temperature used for drying during the following step.

### Preservation Solutions

In some embodiments, the microbial populations to be subjected to one or more preservation challenges are first suspended in a preservation solution. An example preservation solution can include, by way of non-limiting example: an intracellular protectant (e.g., sugars, especially non-reducing sugars; sugar alcohols, such as sorbitol; and/or the like), a pH buffer (e.g., monosodium glutamate, monopotassium phosphate, dipotassium phosphate, and/or the like), a membrane protectant (e.g., polyvinyl-pyrrolidone K-15 and/or the like), as well as components to help with the preservation (e.g., where applicable, sucrose for glass formation, etc.) and quality control (e.g., a redox indicator such as resazurin for use with anaerobic microbes, etc.).

In some embodiments, the intracellular protectant is selected from sorbitol, mannitol, glycerol, maltitol, xylitol, erythritol, and methyl glucoside. In some embodiments, the membrane protectant is selected from sucrose, trehalose, raffinose, polyvinyl pyrrolidone, maltodextrin, and polyethylene glycol. In some embodiments, the preservation solution comprises one or more buffers, e.g., phosphate salts.

In some embodiments, the preservation solutions are tailored to the type of preservation challenges used in the serial preservation methods. One of skill in the art will be familiar with the elements of a preservation solution (e.g., intracellular protectants, a pH buffer, membrane protectants, and the like) and the combinations applicable to each preservation method. For example, a preservation solution used for preservation by foam formation or preservation by vaporization may require higher concentrations of sugars compared to preservation solutions used for other types of preservation challenges.

Exemplary preservation solutions are provided in Tables 3A-Tables 3C in the examples below. Additional preservation solution are described in the art, e.g., U.S. Pat. No. 6,872,357.

### Microbe Sources

In some embodiments, the present disclosure provides methods of improving microbe viability after preservation by subjecting the microbial cultures to serial preservation challenges and preparing a product from the population of viable, preservation challenged microbes present in culture at the conclusion of the preservation challenges. The target microbe population may be any microorganisms suitable for preservation by the methods described herein. As used herein the term “microorganism” should be taken broadly. It includes, but is not limited to, the two prokaryotic domains, Bacteria and Archaea, as well as eukaryotic fungi, protists, and viruses. By way of example, the microorganisms may include species of the genera of: Clostridium, Ruminococcus, Roseburia, Hydrogenoanaerobacterium, Saccharofermentans, Papillibacter, Pelotomaculum, Butyricicoccus, Tannerella, Prevotella, Butyricimonas, Piromyces, Pichia, Candida, Vrystaatia, Orpinomyces, Neocallimastix, and Phyllosticta. The microorganisms may further include species belonging to the family of Lachnospiraceae, and the order of Saccharomycetales. In some embodiments, the microorganisms may include species of any genera disclosed herein.

In one embodiment, the microbes are obtained from animals (e.g., mammals, reptiles, birds, and the like), soil (e.g., rhizosphere), air, water (e.g., marine, freshwater, wastewater sludge), sediment, oil, plants (e.g., roots, leaves, stems), agricultural products, and extreme environments (e.g., acid mine drainage or hydrothermal systems). In a further embodiment, microbes obtained from marine or freshwater environments such as an ocean, river, or lake. In a further embodiment, the microbes can be from the surface of the body of water, or any depth of the body of water (e.g., a deep sea sample).

The microorganisms of the disclosure may be isolated in substantially pure or mixed cultures. They may be concentrated, diluted, or provided in the natural concentrations in which they are found in the source material. For example, microorganisms from saline sediments may be isolated for use in this disclosure by suspending the sediment in fresh water and allowing the sediment to fall to the bottom. The water containing the bulk of the microorganisms may be removed by decantation after a suitable period of settling and either administered to the GI tract of an ungulate, or concentrated by filtering or centrifugation, diluted to an appropriate concentration and administered to the GI tract of an ungulate with the bulk of the salt removed. By way of further example, microorganisms from mineralized or toxic sources may be similarly treated to recover the microbes for application to the ungulate to minimize the potential for damage to the animal.

In another embodiment, the microorganisms are used in a crude form, in which they are not isolated from the source material in which they naturally reside. For example, the microorganisms are provided in combination with the source material in which they reside; for example, fecal matter, cud, or other composition found in the gastrointestinal tract. In this embodiment, the source material may include one or more species of microorganisms.

In some embodiments, a mixed population of microorganisms is used in the methods of the disclosure. In embodiments of the disclosure where the microorganisms are isolated from a source material (for example, the material in which they naturally reside), any one or a combination of a number of standard techniques which will be readily known to skilled persons may be used. However, by way of example, these in general employ processes by which a solid or liquid culture of a single microorganism can be obtained in a substantially pure form, usually by physical separation on the surface of a solid microbial growth medium or by volumetric dilutive isolation into a liquid microbial growth medium. These processes may include isolation from dry material, liquid suspension, slurries or homogenates in which the material is spread in a thin layer over an appropriate solid gel growth medium, or serial dilutions of the material made into a sterile medium and inoculated into liquid or solid culture media.

In some embodiments, the material containing the microorganisms may be pre-treated prior to the isolation process in order to either multiply all microorganisms in the material. Microorganisms can then be isolated from the enriched materials.

The target microbes subjected to the preservation methods described herein can be derived from any sample type that includes a microbial community. For example, samples for use with the methods provided herein encompass without limitation, an animal sample (e.g., mammal, reptile, bird), soil, air, water (e.g., marine, freshwater, wastewater sludge), sediment, oil, plant, agricultural product, plant, soil (e.g., rhizosphere) and extreme environmental sample (e.g., acid mine drainage, hydrothermal systems). In the case of marine or freshwater samples, the sample can be from the surface of the body of water, or any depth of the body water, e.g., a deep sea sample. The water sample, in one embodiment, is an ocean, river, or lake sample.

The animal sample in one embodiment is a body fluid. In another embodiment, the animal sample is a tissue sample. Non-limiting animal samples include tooth, perspiration, fingernail, skin, hair, feces, urine, semen, mucus, saliva, gastrointestinal tract. The animal sample can be, for example, a human, primate, bovine, porcine, canine, feline, rodent (e.g., mouse or rat), equine, or bird sample. In one embodiment, the bird sample comprises a sample from one or more chickens. In another embodiment, the sample is a human sample. The human microbiome comprises the collection of microorganisms found on the surface and deep layers of skin, in mammary glands, saliva, oral mucosa, conjunctiva, and gastrointestinal tract. The microorganisms found in the microbiome include bacteria, fungi, protozoa, viruses, and archaea. Different parts of the body exhibit varying diversity of microorganisms. The quantity and type of microorganisms may signal a healthy or diseased state for an individual. The number of bacteria taxa are in the thousands, and viruses may be as abundant. The bacterial composition for a given site on a body varies from person to person, not only in type, but also in abundance or quantity.

In another embodiment, the sample is a ruminal sample. Ruminants such as cattle rely upon diverse microbial communities to digest their feed. These animals have evolved to use feed with poor nutritive value by having a modified upper digestive tract (reticulorumen or rumen) where feed is held while it is fermented by a community of anaerobic microbes. The rumen microbial community is very dense, with about 3×10¹⁰ microbial cells per milliliter. Anaerobic fermenting microbes dominate in the rumen. The rumen microbial community includes members of all three domains of life: Bacteria, Archaea, and Eukarya. Ruminal fermentation products are required by their respective hosts for body maintenance and growth, as well as milk production (van Houtert (1993). Anim. Feed Sci. Technol. 43, pp. 189-225; Bauman et al. (2011). Annu. Rev. Nutr. 31, pp. 299-319; each incorporated by reference in its entirety for all purposes). Moreover, milk yield and composition has been reported to be associated with ruminal microbial communities (Sandri et al. (2014). Animal 8, pp. 572-579; Palmonari et al. (2010). J. Dairy Sci. 93, pp. 279-287; each incorporated by reference in its entirety for all purposes). Ruminal samples, in one embodiment, are collected via the process described in Jewell et al. (2015). Appl. Environ. Microbiol. 81, pp. 46974710, incorporated by reference herein in its entirety for all purposes.

In another embodiment, the sample is a soil sample (e.g., bulk soil or rhizosphere sample). It has been estimated that 1 gram of soil contains tens of thousands of bacterial taxa, and up to 1 billion bacteria cells as well as about 200 million fungal hyphae (Wagg et al. (2010). Proc Natl. Acad. Sci. USA 111, pp. 5266-5270, incorporated by reference in its entirety for all purposes). Bacteria, actinomycetes, fungi, algae, protozoa, and viruses are all found in soil. Soil microorganism community diversity has been implicated in the structure and fertility of the soil microenvironment, nutrient acquisition by plants, plant diversity and growth, as well as the cycling of resources between above- and below-ground communities. Accordingly, assessing the microbial contents of a soil sample over time and the co-occurrence of active microorganisms (as well as the number of the active microorganisms) provides insight into microorganisms associated with an environmental metadata parameter such as nutrient acquisition and/or plant diversity.

The soil sample in one embodiment is a rhizosphere sample, i.e., the narrow region of soil that is directly influenced by root secretions and associated soil microorganisms. The rhizosphere is a densely populated area in which elevated microbial activities have been observed and plant roots interact with soil microorganisms through the exchange of nutrients and growth factors (San Miguel et al. (2014). Appl. Microbiol. Biotechnol. DOI 10.1007/s00253-014-5545-6, incorporated by reference in its entirety for all purposes). As plants secrete many compounds into the rhizosphere, analysis of the organism types in the rhizosphere may be useful in determining features of the plants which grow therein.

In another embodiment, the sample is a marine or freshwater sample. Ocean water contains up to one million microorganisms per milliliter and several thousand microbial types. These numbers may be an order of magnitude higher in coastal waters with their higher productivity and higher load of organic matter and nutrients. Marine microorganisms are crucial for the functioning of marine ecosystems; maintaining the balance between produced and fixed carbon dioxide; production of more than 50% of the oxygen on Earth through marine phototrophic microorganisms such as Cyanobacteria, diatoms and pico- and nanophytoplankton; providing novel bioactive compounds and metabolic pathways; ensuring a sustainable supply of seafood products by occupying the critical bottom trophic level in marine foodwebs. Organisms found in the marine environment include viruses, bacteria, archaea, and some eukarya. Marine viruses may play a significant role in controlling populations of marine bacteria through viral lysis. Marine bacteria are important as a food source for other small microorganisms as well as being producers of organic matter. Archaea found throughout the water column in the ocean are pelagic Archaea and their abundance rivals that of marine bacteria.

In another embodiment, the sample comprises a sample from an extreme environment, i.e., an environment that harbors conditions that are detrimental to most life on Earth. Organisms that thrive in extreme environments are called extremophiles. Though the domain Archaea contains well-known examples of extremophiles, the domain bacteria can also have representatives of these microorganisms. Extremophiles include: acidophiles which grow at pH levels of 3 or below; alkaliphiles which grow at pH levels of 9 or above; anaerobes such as Spinoloricus cinzia which does not require oxygen for growth; cryptoendoliths which live in microscopic spaces within rocks, fissures, aquifers and faults filled with groundwater in the deep subsurface; halophiles which grow in about at least 0.2M concentration of salt; hyperthermophiles which thrive at high temperatures (about 80-122° C.) such as found in hydrothermal systems; hypoliths which live underneath rocks in cold deserts; lithoautotrophs such as Nitrosomonas europaea which derive energy from reduced mineral compounds like pyrites and are active in geochemical cycling; metallotolerant organisms which tolerate high levels of dissolved heavy metals such as copper, cadmium, arsenic and zinc; oligotrophs which grow in nutritionally limited environments; osmophiles which grow in environments with a high sugar concentration; piezophiles (or barophiles) which thrive at high pressures such as found deep in the ocean or underground; psychrophiles/cryophiles which survive, grow and/or reproduce at temperatures of about −15° C. or lower; radioresistant organisms which are resistant to high levels of ionizing radiation; thermophiles which thrive at temperatures between 45-122° C.; xerophiles which can grow in extremely dry conditions. Polyextremophiles are organisms that qualify as extremophiles under more than one category and include thermoacidophiles (prefer temperatures of 70-80° C. and pH between 2 and 3). The Crenarchaeota group of Archaea includes the thermoacidophiles.

The sample can include microorganisms from one or more domains. For example, in one embodiment, the sample comprises a heterogeneous population of bacteria and/or fungi (also referred to herein as bacterial or fungal strains). For example, the one or more microorganisms can be from the domain Bacteria, Archaea, Eukarya or a combination thereof. Bacteria and Archaea are prokaryotic, having a very simple cell structure with no internal organelles. Bacteria can be classified into gram positive/no outer membrane, gram negative/outer membrane present and ungrouped phyla. Archaea constitute a domain or kingdom of single-celled microorganisms. Although visually similar to bacteria, archaea possess genes and several metabolic pathways that are more closely related to those of eukaryotes, notably the enzymes involved in transcription and translation. Other aspects of archaeal biochemistry are unique, such as the presence of ether lipids in their cell membranes. The Archaea are divided into four recognized phyla: Thaumarchaeota, Aigarchaeota, Crenarchaeota, and Korarchaeota.

The domain of Eukarya comprises eukaryotic organisms, which are defined by membrane-bound organelles, such as the nucleus. Protozoa are unicellular eukaryotic organisms. All multicellular organisms are eukaryotes, including animals, plants, and fungi. The eukaryotes have been classified into four kingdoms: Protista, Plantae, Fungi, and Animalia. However, several alternative classifications exist. Another classification divides Eukarya into six kingdoms: Excavata (various flagellate protozoa); amoebozoa (lobose amoeboids and slime filamentous fungi); Opisthokonta (animals, fungi, choanoflagellates); Rhizaria (Foraminifera, Radiolaria, and various other amoeboid protozoa); Chromalveolata (Stramenopiles (brown algae, diatoms), Haptophyta, Cryptophyta (or cryptomonads), and Alveolata); Archaeplastida/Primoplantae (Land plants, green algae, red algae, and glaucophytes).

Within the domain of Eukarya, fungi are microorganisms that are predominant in microbial communities. Fungi include microorganisms such as yeasts and filamentous fungi as well as the familiar mushrooms. Fungal cells have cell walls that contain glucans and chitin, a unique feature of these organisms. The fungi form a single group of related organisms, named the Eumycota that share a common ancestor. The kingdom Fungi has been estimated at 1.5 million to 5 million species, with about 5% of these having been formally classified. The cells of most fungi grow as tubular, elongated, and filamentous structures called hyphae, which may contain multiple nuclei. Some species grow as unicellular yeasts that reproduce by budding or binary fission. The major phyla (sometimes called divisions) of fungi have been classified mainly on the basis of characteristics of their sexual reproductive structures. Currently, seven phyla are proposed: Microsporidia, Chytridiomycota, Blastocladiomycota, Neocallimastigomycota, Glomeromycota, Ascomycota, and Basidiomycota.

Microorganisms for detection and quantification by the methods described herein can also be viruses. A virus is a small infectious agent that replicates only inside the living cells of other organisms. Viruses can infect all types of life forms in the domains of Eukarya, Bacteria, and Archaea. Virus particles (known as virions) consist of two or three parts: (i) the genetic material which can be either DNA or RNA; (ii) a protein coat that protects these genes; and in some cases (iii) an envelope of lipids that surrounds the protein coat when they are outside a cell. Seven orders have been established for viruses: the Caudovirales, Herpesvirales, Ligamenvirales, Mononegavirales, Nidovirales, Picornavirales, and Tymovirales. Viral genomes may be single-stranded (ss) or double-stranded (ds), RNA or DNA, and may or may not use reverse transcriptase (RT). In addition, ssRNA viruses may be either sense (+) or antisense (−). This classification places viruses into seven groups: I: dsDNA viruses (such as Adenoviruses, Herpesviruses, Poxviruses); H: (+) ssDNA viruses (such as Parvoviruses); III: dsRNA viruses (such as Reoviruses); IV: (+)ssRNA viruses (such as Picornaviruses, Togaviruses); V: (−)ssRNA viruses (such as Orthomyxoviruses, Rhabdoviruses); VI: (+)ssRNA-RT viruses with DNA intermediate in life-cycle (such as Retroviruses); VII: dsDNA-RT viruses (such as Hepadnaviruses).

Microorganisms for detection and quantification by the methods described herein can also be viroids. Viroids are the smallest infectious pathogens known, consisting solely of short strands of circular, single-stranded RNA without protein coats. They are mostly plant pathogens, some of which are of economical importance. Viroid genomes are extremely small in size, ranging from about 246 to about 467 nucleobases.

### Isolated Microbes

As used herein, “isolate”, “isolated”, “isolated microbe”, and like terms, are intended to mean that the one or more microorganisms has been separated from at least one of the materials with which it is associated in a particular environment (for example soil, water, animal tissue). Thus, an “isolated microbe” does not exist in its naturally occurring environment; rather, it is through the various techniques described herein that the microbe has been removed from its natural setting and placed into a non-naturally occurring state of existence. Thus, the isolated strain may exist as, for example, a biologically pure culture, or as spores (or other forms of the strain) in association with an acceptable carrier.

In certain aspects of the disclosure, the isolated microbes exist as isolated and biologically pure cultures. It will be appreciated by one of skill in the art, that an isolated and biologically pure culture of a particular microbe, denotes that said culture is substantially free (within scientific reason) of other living organisms and contains only the individual microbe in question. The culture can contain varying concentrations of said microbe. The present disclosure notes that isolated and biologically pure microbes often necessarily differ from less pure or impure materials. See, e.g. In re Bergstrom, 427 F.2d 1394, (CCPA 1970) (discussing purified prostaglandins), see also, In re Bergy, 596 F.2d 952 (CCPA 1979) (discussing purified microbes), see also, Parke-Davis & Co. v. H.K. Mulford & Co., 189 F. 95 (S.D.N.Y. 1911) (Learned Hand discussing purified adrenaline), aff'd in part, rev'd in part, 196 F. 496 (2d Cir. 1912), each of which are incorporated herein by reference. Furthermore, in some aspects, the disclosure provides for certain quantitative measures of the concentration, or purity limitations, that must be found within an isolated and biologically pure microbial culture. The presence of these purity values, in certain embodiments, is a further attribute that distinguishes the presently disclosed microbes from those microbes existing in a natural state. See, e.g., Merck & Co. v. Olin Mathieson Chemical Corp., 253 F.2d 156 (4th Cir. 1958) (discussing purity limitations for vitamin B12 produced by microbes), incorporated herein by reference.

In some embodiments, the present disclosure provides microbial products prepared by the methods described herein comprising isolated microbial species belonging to taxonomic families of Clostridiaceae, Ruminococcaceae, Lachnospiraceae, Acidaminococcaceae, Peptococcaceae, Porphyromonadaceae, Prevotellaceae, Neocallimastigaceae, Saccharomycetaceae, Phaeosphaeriaceae, Erysipelotrichia, Anaerolinaeceae, Atopobiaceae, Botryosphaeriaceae, Eubacteriaceae, Acholeplasmataceae, Succinivibrionaceae, Lactobacillaceae, Selenomonadaceae, Burkholderiaceae, and Streptococcaceae.

In some embodiments, the present disclosure provides microbial products prepared by the methods described herein comprising isolated microbial species selected from genera of family Clostridiaceae, including Acetanaerobacterium, Acetivibrio, Acidaminobacter, Alkaliphilus, Anaerobacter, Anaerostipes, Anaerotrncus, Anoxynatronum, Bryantella, Butyricicoccus, Caldanaerocella, Caloramator, Caloranaerobacter, Caminicella, Candidatus Arthromitus, Clostridium, Coprobacillus, Dorea, Ethanologenbacterium, Faecalibacterium, Garciella, Guggenheimella, Hespellia, Linmingia, Natronincola, Oxobacter, Parasporobacterium, Sarcina, Soehngenia, Sporobacter, Subdoligranulum, Tepidibacter, Tepidimicrobium, Thermobrachium, Thermohalobacter, and Tindallia.

In some embodiments, the present disclosure provides microbial products prepared by the methods described herein comprising isolated microbial species selected from genera of family Ruminococcaceae, including Runinococcus, Acetivibrio, Sporobacter, Anaerofilium, Papillibacter, Oscillospira, Gemmiger, Faecalibacterium, Fastidiosipila, Anaerotrncus, Ethanolingenens, Acetanaerobacterium, Subdoligranulum, Hydrogenoanaerobacterium, and Candidadus soleaferrea.

In some embodiments, the present disclosure provides microbial products prepared by the methods described herein comprising isolated microbial species selected from genera of family Lachnospiraceae, including Butyrivibrio, Roseburia, Lachnospira, Acetitomaculum, Coprococcus, Johnsonella, Catonella, Pseudobutyrivibrio, Syntrophococcus, Sporobacterium, Paraspxorobacterium, Lachnobacterium, Shuttleworthia, Dorea, Anaerostipes, Hespellia, Marvinbryantia, Oribacterium, Moryella, Blautia, Robinsoniella, Cellulosilyticum, Lachnoanaerobaculum, Stomatobaculum, Fusicatenibacter, Acetatifactor, and Eisenbergiella.

In some embodiments, the present disclosure provides microbial products prepared by the methods described herein comprising isolated microbial species selected from genera of family Acidaminococcaceae, including Acidaminococcus, Phascolarctobacterium, Succiniclasticum, and Succinispira.

In some embodiments, the present disclosure provides microbial products prepared by the methods described herein comprising isolated microbial species selected from genera of family Peptococcaceae, including Desulfotomaculum, Peptococcus, Desulftobacterium, S ntrophobotulus, Dehalobacter, Sporotomaculum, Desulfosporosinus, Desulfonispora. Pelotomaculum, Thermincola, Cryptanaerobacter, Desulftibacter, Candidatus Desulforudis, Desulfuirispora, and Desulfitospora.

In some embodiments, the present disclosure provides microbial products prepared by the methods described herein comprising isolated microbial species selected from genera of family Porphyromonadaceae, including Porphyromonas, Dysgonomonas, Tannerella, Odoribacter, Proteiniphilum, Petrinonas, Paludibacter, Parabacteroides, Barnesiella, Candidatus Vestibaculum, Butyricimonas, Macellibacteroides, and Coprobacter.

In some embodiments, the present disclosure provides microbial products prepared by the methods described herein comprising isolated microbial species selected from genera of family Anaerolinaeceae including Anaerolinea, Bellilinea, Leptolinea, Levilinea, Longilinea, Ornatilinea, and Pelolinea.

In some embodiments, the present disclosure provides microbial products prepared by the methods described herein comprising isolated microbial species selected from genera of family Atopobiaceae including Atopbium and Olsenella.

In some embodiments, the present disclosure provides microbial products prepared by the methods described herein comprising isolated microbial species selected from genera of family Eubacteriaceae including Acetobacterium, Alkalibacter, Alkalibaculum, Aminicella, Anaerofustis, Eubacterium, Garciella, and Pseudoramibacter.

In some embodiments, the present disclosure provides microbial products prepared by the methods described herein comprising isolated microbial species selected from genera of family Acholeplasmataceae including Acholeplasma.

In some embodiments, the present disclosure provides microbial products prepared by the methods described herein comprising isolated microbial species selected from genera of family Succinivibrionaceae including Anaerobiospirillum, Ruminobacter, Succinatimonas, Succinimonas, and Succinivibrio.

In some embodiments, the present disclosure provides microbial products prepared by the methods described herein comprising isolated microbial species selected from genera of family Lactobacillaceae including Lactobacillus, Paralactobacillus, Pediococcus, and Sharpea.

In some embodiments, the present disclosure provides microbial products prepared by the methods described herein comprising isolated microbial species selected from genera of family Selenomonadaceae including Anaerovibrio, Centipeda, Megamonas, Mitsuokella, Pectinatus, Propionispira, Schwartzia, Selenomonas, and Zymophilus.

In some embodiments, the present disclosure provides microbial products prepared by the methods described herein comprising isolated microbial species selected from genera of family Burkholderiaceae including Burkholderia, Chitinimonas, Cupriavidus, Lautropia, Limnobacter, Pandoraea, Paraburkholderia, Paucimonas, Polynucleobacter, Ralstonia, Thermothrix, and Wautersia.

In some embodiments, the present disclosure provides microbial products prepared by the methods described herein comprising isolated microbial species selected from genera of family Streptococcaceae including Lactococcus, Lactovum, and Streptococcus.

In some embodiments, the present disclosure provides microbial products prepared by the methods described herein comprising isolated microbial species selected from genera of family Anaerolinaeceae including Aestuariimicrobium, Arachnia, Auraticoccus, Brooklawnia, Friedmanniella, Granulicoccus, Luteococcus, Mariniluteicoccus, Microlunatus, Micropruina, Naumannella, Propionibacterium, Propionicicella, Propioniciclaw, Propioniferax, Propionimicrobium, and Tessaracoccus.

In some embodiments, the present disclosure provides microbial products prepared by the methods described herein comprising isolated microbial species selected from genera of family Prevotellaceae, including Paraprevotella, Prevotella, hallella, Xylanibacter, and Alloprevotella.

In some embodiments, the present disclosure provides microbial products prepared by the methods described herein comprising isolated microbial species selected from genera of family Neocallimastigaceae, including Anaeromyces, Caecomyces, Cyllanmyces, Neocallimastix, Orpinomyces, and Piromyces.

In some embodiments, the present disclosure provides microbial products prepared by the methods described herein comprising isolated microbial species selected from genera of family Saccharomycetaceae, including Brettanoryces, Candida, Citeromyces, Cyniclomyces, Debaryomyces, Issatchenkia, Kazachstania (syn. Arxiozyma), Kluyveromyces, Komagataella, Kuraishia, Lachancea, Lodderomyces, Nakaseomyces, Pachysolen, Pichia, Saccharomyces, Spathaspora, Tetrapisispora, Vanderwaltozyma, Torulaspora, Williopsis, Zygosaccharomyces, and Zygotondaspora.

In some embodiments, the present disclosure provides microbial products prepared by the methods described herein comprising isolated microbial species selected from genera of family Erysipelotrichaceae, including Erysipelothrix, Solobacterium, Turicibacter, Faecalibaculum, Faecalicoccus, Faecalitalea, Holderanella, Holdemania, Dielma, Eggerthia, Erysipelatoclostridium, Allobacterium, Breznakia, Bulleidia, Catenibacterium, Catenisphaera, and Coprobacillus.

In some embodiments, the present disclosure provides microbial products prepared by the methods described herein comprising isolated microbial species selected from genera of family Phaeosphaeriaceae, including Barria, Bricookea, Carinispora, Chaetoplea, Eudarluca, Hadrospora, Isthmosporella, Katumotoa, Lautitia, Metameris, Mixtura, Neophaeosphaeria, Nodulosphaeria, Ophiosphaerella, Phaeosphaerns, Phaeosphaeriopsis, Setomelanomma, Stagonospora, Teratosphaeria, and Wilmia.

In some embodiments, the present disclosure provides microbial products prepared by the methods described herein comprising isolated microbial species selected from genera of family Botryosphaeriaceae, including Amarenomyces, Aplosporella, Auerswaldiella, Botryosphaeria, Dichomera, Diplodia, Discochora, Dothidothia, Dothiorella, Fusicoccum, Granulodiplodia, Guignardia, Lasiodiplodia, Leptodothiorella, Leptodothiorella, Leptoguignardia, Macrophoma, Macrophomina, Nattrassia, Neodeightonia, NeolMsicocum, Neoscytalidium, Otthia, Phaeobotryosphaeria, Phomatosphaeropsis, Phyllosticta, Pseudofsicoccum, Saccharata, Sivanesania, and Thyrostroma.

In some embodiments, the present disclosure provides microbial products prepared by the methods described herein comprising isolated microbial species selected from genera of: Clostridium, Ruminococcus, Roseburia, Hydrogenoanaerobacterium, Saccharofermentans, Papillibacter, Pelotomaculum, Butyricicoccus, Tannerella, Prevotella, Butyricimonas, Piromyces, Candida, Vrystaatia, Orpinomyces, Neocallirastix, and Phyllosticta. In further embodiments, the disclosure provides microbial products produced by the methods described herein comprising isolated microbial species belonging to the family of Lachnospiraceae, and the order of Saccharomycetales. In further embodiments, the disclosure provides microbial products produced by the methods described herein comprising isolated microbial species of Candida xylopsoci, Vrystaatia aloeicola, and Phyllosticta capitalensis.

In some embodiments, the present disclosure provides microbial products prepared by the methods described herein comprising isolated microbial species selected from a Clostridium spp. bacterium, a Siccinivibrio spp. bacterium, a Caecoryces spp. fungus, a Pichia spp. fungus, a Butyrivibio spp. bacterium, an Orpinomyces spp. fungus, a Piromyces spp. fungus, a Bacillus spp. bacterium, a Lactobacillus spp. bacterium, a Prevotella spp. bacterium, a Syntrophococcus spp. bacterium, or a Ruminococcus spp. bacterium. In some embodiments, the present disclosure provides microbial products prepared by the methods described herein comprising isolated microbial species selected from genera of family Lachnospiraceae.

In some embodiments, the isolated microbial strains in the products described herein have been genetically modified. In some embodiments, the genetically modified or recombinant microbes comprise polynucleotide sequences which do not naturally occur in said microbes. In some embodiments, the microbes may comprise heterologous polynucleotides. In further embodiments, the heterologous polynucleotides may be operably linked to one or more polynucleotides native to the microbes.

In some embodiments, the heterologous polynucleotides may be reporter genes or selectable markers. In some embodiments, reporter genes may be selected from any of the family of fluorescence proteins (e.g., GFP, RFP, YFP, and the like), β-galactosidase, or luciferase. In some embodiments, selectable markers may be selected from neomycin phosphotransferase, hygromycin phosphotransferase, aminoglycoside adenyltransferase, dihydrofolate reductase, acetolactase synthase, bromoxynil nitrilase, β-glucuronidase, dihydrogolate reductase, and chloramphenicol acetyltransferase. In some embodiments, the heterologous polynucleotide may be operably linked to one or more promoter.

In some embodiments, the isolated microbes are identified by ribosomal nucleic acid sequences. Ribosomal RNA genes (rDNA), especially the small subunit ribosomal RNA genes, i.e., 18S rRNA genes (18S rDNA) in the case of eukaryotes and 16S rRNA (16S rDNA) in the case of prokaryotes, have been the predominant target for the assessment of organism types and strains in a microbial community. However, the large subunit ribosomal RNA genes, 28S rDNAs, have been also targeted. rDNAs are suitable for taxonomic identification because: (i) they are ubiquitous in all known organisms; (ii) they possess both conserved and variable regions; (iii) there is an exponentially expanding database of their sequences available for comparison. In community analysis of samples, the conserved regions serve as annealing sites for the corresponding universal PCR and/or sequencing primers, whereas the variable regions can be used for phylogenetic differentiation. In addition, the high copy number of rDNA in the cells facilitates detection from environmental samples.

The internal transcribed spacer (ITS), located between the 18S rDNA and 28S rDNA, has also been targeted. The ITS is transcribed but spliced away before assembly of the ribosomes. The ITS region is composed of two highly variable spacers, ITS1 and ITS2, and the intercalary 5.8S gene. This rDNA operon occurs in multiple copies in genomes. Because the ITS region does not code for ribosome components, it is highly variable. In some embodiments, the unique RNA marker can be an mRNA marker, an siRNA marker, or a ribosomal RNA marker.

The primary structure of major rRNA subunit 16S comprise a particular combination of conserved, variable, and hypervariable regions that evolve at different rates and enable the resolution of both very ancient lineages such as domains, and more modern lineages such as genera. The secondary structure of the 16S subunit include approximately 50 helices which result in base pairing of about 67% of the residues. These highly conserved secondary structural features are of great functional importance and can be used to ensure positional homology in multiple sequence alignments and phylogenetic analysis. Over the previous few decades, the 16S rRNA gene has become the most sequenced taxonomic marker and is the cornerstone for the current systematic classification of bacteria and archaea (Yarza et al. 2014. Nature Rev. Micro. 12:635-45).

In some embodiments, a sequence identity of 94.5% or lower for two 16 rRNA genes is strong evidence for distinct genera, 86.5% or lower is strong evidence for distinct families, 82% or lower is strong evidence for distinct orders, 78.5% is strong evidence for distinct classes, and 75% or lower is strong evidence for distinct phyla. The comparative analysis of 16S rRNA gene sequences enables the establishment of taxonomic thresholds that are useful not only for the classification of cultured microorganisms but also for the classification of the many environmental sequences. Yarza et al 2014. Nature Rev. Micro. 12:635-45).

Exemplary isolated microbes that can be preserved and incorporated into a product according to the methods described herein are provided below in Table 2.

TABLE 2
Exemplary Isolated Microbes

| Predicted | BLAST | | 16S or ITS Nucleic Acid | SEQ |
|---|---|---|---|---|
| Taxa | Taxonomic Hit | Ref ID: | Sequence | ID: |
| Clostridium | Clostridium | Ascusb_3138; | AGAGTTTGATCCTGGCTCAGGAC | 1 |
| sensu stricto | butyricum | DY-20 | GAACGCTGGCGGCGTGCTTAACA | |
| | | | CATGCAAGTCGAGCGATGAAGTT | |
| | | | CCTTCGGGAATGGATTAGCGGCG | |
| | | | GACGGGTGAGTAACACGTGGGTA | |
| | | | ACCTGCCTCATAGAGGGGAATAG | |
| | | | CCTTTCGAAAGGAAGATTAATAC | |
| | | | CGCATAAGATTGTAGCACCGCAT | |
| | | | GGTGCAGCAATTAAAGGAGTAAT | |
| | | | CCGCTATGAGATGGACCC | |
| Candida | Pichia | Ascusf_15; | TCCTCCGCTTATTGATATGCTTA | 2 |
| xylopsoc | kudriazevii | DY-21 | AGTTCAGCGGGTATTCCTACCTG | |
| | | | ATTTGAGGTCGAGCTTTTTGTTG | |
| | | | TCTCGCAACACTCGCTCTCGGCC | |
| | | | GCCAAGCGTCCCTGAAAAAAAGT | |
| | | | CTAGTTCGCTCGGCCAGCTTCGC | |
| | | | TCCCTTTCAGGCGAGTCGCAGCT | |
| | | | CCGACGCTCTTTACACGTCGTCC | |
| | | | GCTCCGCTCCCCCAACTCTGCGC | |
| | | | ACGCGCAAGATGGAAACG | |
| Clostridium | Ruminococcus | Ascusb_5; | AGAGTTTGATCCTGGCTCAGGAT | 3 |
| IV | bromii | DY-10 | GAACGCTGGCGGCGTGCCTAACA | |
| | | | CATGCAAGTCGAACGGAACTTCT | |
| | | | TTGACAGAATTCTTCGGAAGGAA | |
| | | | GTTGATTAAGTTTAGTGGCGGAC | |
| | | | GGGTGAGTAACGCGTGAGTAACC | |
| | | | TGCCTTTGAGAGGGGAATAACTT | |
| | | | CCCGAAAGGGATGCTAATACCGC | |
| | | | ATAAAGCATAGAAGTCGCATGGC | |
| | | | TTTTATGCCAAAGATTTA | |
| Bacillus | Bacillus subtilis | Ascusbbr_33(A); | AGATTTGATCATGGCTCAGGACG | 4 |
| | | BR-11 | AACGCTGGCGGCGTGCCTAATAC | |
| | | | ATGCAAGTCGAGCGGACAGATGG | |
| | | | GAGCTTGCTCCCTGATGTTAGCG | |
| | | | GCGGACGGGTGAGTAACACGTGG | |
| | | | GTAACCTGCCTGTAAGACTGGGA | |
| | | | TAACTCCGGGAAACCGGGGCTAA | |
| | | | TACCGGATGGTTGTCTGAACCGC | |
| | | | ATGGTTCAGACATAAAAGGTGGC | |
| | | | TTCGGCTACCACTTACA | |
| Clostridium | Clostridium | Ascusbbr_105932; | AGAGTTTGATCCTGGCTCAGGAT | 5 |
| | saccharolyticum | BR-21 | GAACGCTGGCGGCGTGCTTAACA | |
| | | | CATGCAAGTCGAGCGAAGCAGTT | |
| | | | TTAAGGAAGTTTTCGGATGGAAT | |
| | | | TAAAATTGACTTAGCGGCGGACG | |
| | | | GGTGAGTAACGCGTGGGTAACCT | |
| | | | GCCTCATACAGGGGGATAACAGT | |
| | | | TAGAAATGACTGCTAATACCGCA | |
| | | | TAAGCGCACAGTGCTGCATAGCA | |
| | | | CAGTGTGAAAAACTCCG | |
| Clostridium | Clostridium | Ascusbbr_2676; | AGAGTTTGATCATGGCTCAGGAC | 6 |
| | beijerinckii | BR-67 | GAACGCTGGCGGCGTGCTTAACA | |
| | | | CATGCAAGTCGAGCGATGAAGTT | |
| | | | CCTTCGGGAACGGATTAGCGGCG | |
| | | | GACGGGTGAGTAACACGTGGGTA | |
| | | | ACCTGCCTCATAGAGGGGAATAG | |
| | | | CCTTCCGAAAGGAAGATTAATAC | |
| | | | CGCATAAGATTGTAGTTTCGCAT | |
| | | | GAAACAGCAATTAAAGGAGTAAT | |
| | | | CCGCTATGAGATGGACC | |
| Lactobacillus | Lactobacillus | Ascusbbr_5796 | AGATTTGCTCCTGGCTCAGGACG | 7 |
| | crispatus | (A); BR-16 | AACGCTGGCGGCGTGCCTAATAC | |
| | | | ATGCAAGTCGAGCGAGCGGAACT | |
| | | | AACAGATTTACTTCGGTAATGAC | |
| | | | GTTAGGAAAGCGAGCGGCGGATG | |
| | | | GGTGAGTAACACGTGGGGAACCT | |
| | | | GCCCCATAGTCTGGGATACCACT | |
| | | | TGGAAACAGGTGCTAATACCGGA | |
| | | | TAAGAAAGCAGATCGCATGATCA | |
| | | | GCTTTTAAAAGGCGGCG | |
| Lactobacillus | Lactobacillus | Ascusbbr_5796 | AGAGTTTGATCATGGCTCAGGAC | 8 |
| | crispatus | (B); BR-16 | GAACGCTGGCGGCGTGCCTAATA | |
| | | | CATGCAAGTCGAGCGAGCGGAAC | |
| | | | TAACAGATTTACTTCGGTAATGA | |
| | | | CGTTAGGAAAGCGAGCGGCGGAT | |
| | | | GGGTGAGTAACACGTGGGGAACC | |
| | | | TGCCCCATAGTCTGGGATACCAC | |
| | | | TTGGAAACAGGTGCTAATACCGG | |
| | | | ATAAGAAAGCAGATCGCATGATC | |
| | | | AGCTTTTAAAAGGCGGC | |
| Lactobacillus | Lactobacillus | Ascusbbr_5796 | AGAGTTTGATCCTGGCTCAGGAC | 9 |
| | crispatus | (C); BR-16 | GAACGCTGGCGGCGTGCCTAATA | |
| | | | CATGCAAGTCGAGCGAGCGGAAC | |
| | | | TAACAGATTTACTTCGGTAATGA | |
| | | | CGTTAGGAAAGCGAGCGGCGGAT | |
| | | | GGGTGAGTAACACGTGGGGAACC | |
| | | | TGCCCCATAGTCTGGGATACCAC | |
| | | | TTGGAAACAGGTGCTAATACCGG | |
| | | | ATAAGAAAGCAGATCGCATGATC | |
| | | | AGCTTTTAAAAGGCGGCG | |
| Prevotella | Prevotella | Ascusbbf_4; | AGAGTTTGATCCTGGCTCAGGAT | 10 |
| | albensis | BY-41 | GAACGCTAGCTACAGGCTTAACA | |
| | | | CATGCAAGTCGAGGGGAAACGAC | |
| | | | ATAGAGTGCTTGCACTTTATGGG | |
| | | | CGTCGACCGGCGAATGGGTGAGT | |
| | | | AACGCGTATCCAACCTGCCCTTG | |
| | | | ACCGAGGGATAGCCCAGTGAAAA | |
| | | | CTGAATTAATACCTCATGTTCTC | |
| | | | CTCAGACGGCATCAGACGAGGAG | |
| | | | CAAAGATTAATCGGTCAA | |
| Succinivibrio | Succinivibrio | Ascusbbf_154; | AGAGTTTGATCATGGCTCAGATT | 11 |
| | dextrinosolvens | BF-53 | GAACGCTGGCGGCAGGCCTAATA | |
| | | | CATGCAAGTCGAACGGTAACATA | |
| | | | GGAAAAGCTTGCTTTTCCTGATG | |
| | | | ACGAGTGGCGGACGGGTGAGTAA | |
| | | | AGTTTGGGAAGCTACCTGATAGA | |
| | | | GGGGGACAACAGTTGGAAACGAC | |
| | | | TGCTAATACCGCATACAGCCTGA | |
| | | | GGGTGAAAGCAGCAATGCGCTAT | |
| | | | CAGATGCGCCCAAATGGG | |
| Lachnospiraceae | | Ascusbbf_876; | AGAGTTTGATCCTGGCTCAGGAT | 12 |
| | | BF-65 | GAACGCTGGCGGCGTGCCTAACA | |
| | | | CATGCAAGTCGAGCGGAGTGAAG | |
| | | | AGAGCTTGCTTTTTTCACTTAGC | |
| | | | GGCGGATGGGTGAGGAACGCGTG | |
| | | | GGGAACCTGCCTCTCACAGGGGG | |
| | | | ATAACAGCTGGAAACGGCTGTTA | |
| | | | ATACCGCATATGCACACAGTGCC | |
| | | | GCATGGCACAGGGTGGAAAGAAA | |
| | | | TTCGGTGAGAGATGGACC | |

### Microbial Ensembles

In some aspects, the disclosure provides microbial products produced by the methods described herein and comprising microbial ensembles comprising a combination of at least two viability-enhanced microbes. In certain embodiments, the ensembles of the present disclosure comprise two microbes, or three microbes, or four microbes, or five microbes, or six microbes, or seven microbes, or eight microbes, or nine microbes, or ten or more microbes. Said microbes of the ensembles are different microbial species, or different strains of a microbial species.

As used herein, “microbial ensemble” refers to a composition comprising one or more active microbes that does not naturally exist in a naturally occurring environment and/or at ratios or amounts that do not exist in a nature. For example, a microbial ensemble (also synthetic ensemble and/or bioensemble) or aggregate could be formed from one or more isolated microbe strains, along with an appropriate medium or carrier. Microbial ensembles can be applied or administered to a target, such as a target environment, population, individual, animal, and/or the like.

In certain aspects of the disclosure, microbial ensembles are or are based on one or more isolated microbes that exist as isolated and biologically pure cultures.

In some aspects, the disclosure provides microbial products produced by the methods described herein and comprising microbial ensembles, wherein the microbial ensemble comprises at least two isolated microbial species selected from a Clostridium spp. bacterium, a Succinivibrio spp. bacterium, a Caecomyces spp. fungus, a Pichia spp. fungus, a Butyrivibio spp. bacterium, an Orpinomyces spp. fungus, a Piromyces spp. fungus, a Bacillus spp. bacterium, a Lactobacillus spp. bacterium, a Prevotella spp. bacterium, a Syntrophococcus spp. bacterium, or a Ruminococcus spp. bacterium. Exemplary species are provided above in Table 2.

In some aspects, the disclosure provides microbial products produced by the methods described herein and comprising microbial ensembles, wherein the microbial ensemble comprises a Clostridium spp. comprising a 16S rRNA sequence with at least 97%, 98%, or 99% sequence identity to SEQ ID NO: 1, SEQ ID NO: 3, SEQ ID NO: 5, or SEQ ID NO: 6. In some aspects, the microbial ensemble comprises a Clostridium spp. comprising a 16S rRNA sequence comprising or consisting of SEQ ID NO: 1, SEQ ID NO: 3, SEQ ID NO: 5, or SEQ ID NO: 6. In some aspects, the disclosure provides microbial products produced by the methods described herein and comprising microbial ensembles, wherein the microbial ensemble comprises a species from the family Lachnospiraceae comprising a 16S rRNA sequence with at least 97%, 98%, or 99% sequence identity to SEQ ID NO: 12. In some aspects, the microbial ensemble comprises a species from the family Lachnospiraceae comprising a 16S rRNA sequence comprising or consisting SEQ ID NO: 12.

In some aspects, the disclosure provides microbial products produced by the methods described herein and comprising microbial ensembles, wherein the microbial ensemble comprises a Succinivibrio spp. comprises a 16S rRNA sequence comprising at least 97%, 98%, or 99% sequence identity to SEQ ID NO: 11. In some aspects, the microbial ensemble comprises a Succinivibrio spp. comprising a 16S rRNA sequence comprising or consisting of SEQ ID NO: 11.

In some aspects, the disclosure provides microbial products produced by the methods described herein and comprising microbial ensembles, wherein the microbial ensemble comprises a Pichia spp. comprises an ITS sequence comprising at least 97%, 98%, or 99% sequence identity to SEQ ID NO: 2. In some aspects, the microbial ensemble comprises a Pichia spp. comprising an ITS sequence comprising or consisting of SEQ ID NO: 2.

In some aspects, the disclosure provides microbial products produced by the methods described herein and comprising microbial ensembles, wherein the microbial ensemble comprises a Bacillus spp. comprises a 16S rRNA sequence comprising at least 97%, 98%, or 99% sequence identity to SEQ ID NO: 4. In some aspects, the microbial ensemble comprises a Bacillus spp. comprising or consisting of SEQ ID NO: 4. In some aspects, the disclosure provides microbial products produced by the methods described herein and comprising microbial ensembles, wherein the microbial ensemble comprises a Lactobacillus spp. comprises a 16S rRNA sequence comprising at least 97%, 98%, or 99% sequence identity to SEQ ID NO: 7, SEQ ID NO: 8, or SEQ ID NO: 9. In some aspects, the microbial ensemble comprises a Lactobacillus spp. comprising a 16S rRNA sequence comprising or consisting of SEQ ID NO: 7, SEQ ID NO: 8, or SEQ ID NO: 9.

In some aspects, the disclosure provides microbial products produced by the methods described herein and comprising microbial ensembles, wherein the microbial ensemble comprises a Prevotella spp. comprises a 16S rRNA sequence comprising at least 97%, 98%, or 99% sequence identity to SEQ ID NO: 10. In some aspects, the microbial ensemble comprises a Prevotella spp. comprising a 16S rRNA sequence comprising or consisting of SEQ ID NO: 10.

In some aspects, the disclosure provides microbial products produced by the methods described herein and comprising microbial ensembles, wherein the microbial ensemble comprises Clostridium butyricum comprising at least 97%, 98%, or 99% sequence identity to SEQ ID NO: 1 and Pichia kudriazevii comprising at least 97%, 98%, or 99% sequence identity to SEQ ID NO: 2.

In some aspects, the disclosure provides microbial products produced by the methods described herein and comprising microbial ensembles, wherein the microbial ensemble comprises a Clostridium spp. comprising at least 97%, 98%, or 99% sequence identity to SEQ ID NO: 5, a Clostridium spp. comprising at least 97%, 98%, or 99% sequence identity to SEQ ID NO: 6, and a Lactobacillus spp. comprising at least 97%, 98%, or 99% sequence identity to SEQ ID NO: 7. In some aspects, the disclosure provides microbial products produced by the methods described herein and comprising microbial ensembles, wherein the microbial ensemble comprises a Clostridium spp. comprising at least 97%, 98%, or 99% sequence identity to SEQ ID NO: 5, and a Clostridium spp. comprising at least 97%, 98%, or 99% sequence identity to SEQ ID NO: 6.

In some aspects, the disclosure provides microbial products produced by the methods described herein and comprising microbial ensembles, wherein the microbial ensemble comprises a Prevotella spp. comprising at least 97%, 98%, or 99% sequence identity to SEQ ID NO: 10, a Succinivibrio spp. comprising at least 97%, 98%, or 99% sequence identity to SEQ ID NO: 11, and a Lachnospiraceae species comprising at least 97%, 98%, or 99% sequence identity to SEQ ID NO: 12.

In some aspects, the disclosure provides microbial products produced by the methods described herein and comprising microbial ensembles, wherein the microbial ensemble comprises at least two isolated microbial species selected from a genera of: Clostridium, Ruminococcus, Roseburia, Hydrogenoanaerobacterium, Saccharofermentans, Papillibacter, Pelotomaculum, Butyricicoccus, Tannerella, Prevotella, Butyricimonas, Piromyces, Pichia, Candida, Vrystaatia, Orpinomyces, Neocallimastix, and Phyllosticta.

### Microbial Strains

Microbes can be distinguished into a genus based on polyphasic taxonomy, which incorporates all available phenotypic and genotypic data into a consensus classification (Vandamme et al. 1996. Polyphasic taxonomy, a consensus approach to bacterial systematics. Microbiol Rev 1996, 60:407-438). One accepted genotypic method for defining species is based on overall genomic relatedness, such that strains which share approximately 70% or more relatedness using DNA-DNA hybridization, with 5° C. or less ΔTₘ (the difference in the melting temperature between homologous and heterologous hybrids), under standard conditions, are considered to be members of the same species. Thus, populations that share greater than the aforementioned 70% threshold can be considered to be variants of the same species. Another accepted genotypic method for defining species is to isolate marker genes of the present disclosure, sequence these genes, and align these sequenced genes from multiple isolates or variants. The microbes are interpreted as belonging to the same species if one or more of the sequenced genes share at least 97% sequence identity.

Isolated microbes can be matched to their nearest taxonomic groups by utilizing classification tools of the Ribosomal Database Project (RDP) for 16s rRNA sequences and the User-friendly Nordic ITS Ectomycorrhiza (UNITE) database for ITS rRNA sequences. Examples of matching microbes to their nearest taxa may be found in Lan et al. (2012. PLOS one. 7(3):e32491), Schloss and Westcott (2011. Appl. Environ. Microbiol. 77(10):3219-3226), and Koljalg et al. (2005. New Phytologist. 166(3):1063-1068). The 16S or 18S rRNA sequences or ITS sequences are often used for making distinctions between species and strains, in that if one of the aforementioned sequences share less than a specified percent sequence identity from a reference sequence, then the two organisms from which the sequences were obtained are said to be of different species or strains. Comparisons may also be made with 23S rRNA sequences against reference sequences.

Thus, one could consider microbes to be of the same species, if they share at least 80%, 85%, 90%, 95%, 97%, 98%, or 99% sequence identity across the 16S or 18S rRNA sequence, or the ITS1 or ITS2 sequence. Further, one could define microbial strains of a species, as those that share at least 80%, 85%, 90%, 95%, 97%, 98%, or 99% sequence identity across the 16S or 18S rRNA sequence, or the ITS1 or ITS2 sequence.

In one embodiment, microbial strains of the present disclosure include those that comprise polynucleotide sequences that share at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any one of SEQ ID NOs:1-12. In a further embodiment, microbial strains of the present disclosure include those that comprise polynucleotide sequences that share at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any one of SEQ ID NOs: 1-12.

Unculturable microbes often cannot be assigned to a definite species in the absence of a phenotype determination, the microbes can be given a candidatus designation within a genus provided their 16S or 18S rRNA sequences or ITS sequences subscribes to the principles of identity with known species.

One approach is to observe the distribution of a large number of strains of closely related species in sequence space and to identify clusters of strains that are well resolved from other clusters. This approach has been developed by using the concatenated sequences of multiple core (house-keeping) genes to assess clustering patterns, and has been called multilocus sequence analysis (MLSA) or multilocus sequence phylogenetic analysis. MLSA has been used successfully to explore clustering patterns among large numbers of strains assigned to very closely related species by current taxonomic methods, to look at the relationships between small numbers of strains within a genus, or within a broader taxonomic grouping, and to address specific taxonomic questions. More generally, the method can be used to ask whether bacterial species exist—that is, to observe whether large populations of similar strains invariably fall into well-resolved clusters, or whether in some cases there is a genetic continuum in which clear separation into clusters is not observed.

In order to more accurately make a determination of genera, a determination of phenotypic traits, such as morphological, biochemical, and physiological characteristics can be made for comparison with a reference genus archetype. The colony morphology can include color, shape, pigmentation, production of slime, etc. Features of the cell are described as to shape, size, Gram reaction, extracellular material, presence of endospores, flagella presence and location, motility, and inclusion bodies. Biochemical and physiological features describe growth of the organism at different ranges of temperature, pH, salinity, and atmospheric conditions, growth in presence of different sole carbon and nitrogen sources. One of skill should be reasonably apprised as to the phenotypic traits that define the genera of the present disclosure.

In one embodiment, the microbes taught herein were identified utilizing 16S rRNA gene sequences and ITS sequences. It is known in the art that 16S rRNA contains hypervariable regions that can provide species/strain-specific signature sequences useful for bacterial identification, and that ITS sequences can also provide species/strain-specific signature sequences useful for fungal identification.

Phylogenetic analysis using the rRNA genes and/or ITS sequences are used to define “substantially similar” species belonging to common genera and also to define “substantially similar” strains of a given taxonomic species. Furthermore, physiological and/or biochemical properties of the isolates can be utilized to highlight both minor and significant differences between strains that could lead to advantageous behavior in ruminants.

Compositions of the present disclosure may include combinations of fungal spores and bacterial spores, fungal spores and bacterial vegetative cells, fungal vegetative cells and bacterial spores, fungal vegetative cells and bacterial vegetative cells. In some embodiments, compositions of the present disclosure comprise bacteria only in the form of spores. In some embodiments, compositions of the present disclosure comprise bacteria only in the form of vegetative cells. In some embodiments, compositions of the present disclosure comprise bacteria in the absence of fungi. In some embodiments, compositions of the present disclosure comprise fungi in the absence of bacteria.

Bacterial spores may include endospores and akinetes. Fungal spores may include statismospores, ballistospores, autospores, aplanospores, zoospores, mitospores, megaspores, microspores, meiospores, chlamydospores, urediniospores, teliospores, oospores, carpospores, tetraspores, sporangiospores, zygospores, ascospores, basidiospores, ascospores, and asciospores.

### Microbial Products

In some embodiments, the present disclosure provides a product prepared by the serial preservation methods described herein and comprising a population of preserved viability-enhanced microbial cells. In some embodiments, the microbial products prepared by the methods described herein comprise one or more viability-enhanced microbe(s) and an acceptable carrier. In a further embodiment, the viability-enhanced microbe(s) is encapsulated. In a further embodiment, the encapsulated viability-enhanced microbe(s) comprises a polymer. In a further embodiment, the polymer may be selected from a saccharide polymer, agar polymer, agarose polymer, protein polymer, sugar polymer, and lipid polymer.

In some embodiments, the acceptable carrier is selected from the group consisting of edible feed grade material, mineral mixture, water, glycol, molasses, and corn oil. In some embodiments, the at least two microbial strains forming the microbial ensemble are present in the composition at 10² to 10¹⁵ cells per gram of said composition. In some embodiments, the composition may be mixed with a feed composition.

In some embodiments, the microbial products of the present disclosure are administered to an animal. In some embodiments, the composition is administered at least once per day. In a further embodiment, the composition is administered at least once per month. In a further embodiment, the composition is administered at least once per week. In a further embodiment, the composition is administered at least once per hour.

In some embodiments, the administration comprises injection of the composition into the rumen. In some embodiments, the composition is administered anally. In further embodiments, anal administration comprises inserting a suppository into the rectum. In some embodiments, the composition is administered orally. In some aspects, the oral administration comprises administering the composition in combination with the animal's feed, water, medicine, or vaccination. In some aspects, the oral administration comprises applying the composition in a gel or viscous solution to a body part of the animal, wherein the animal ingests the composition by licking. In some embodiments, the administration comprises spraying the composition onto the animal, and wherein the animal ingests the composition. In some embodiments, the administration occurs each time the animal is fed. In some embodiments, the oral administration comprises administering the composition in combination with the animal feed.

In some embodiments, the microbial products of the present disclosure include ruminant feed, such as cereals (barley, maize, oats, and the like); starches (tapioca and the like); oilseed cakes; and vegetable wastes. In some embodiments, the microbial products include vitamins, minerals, trace elements, emulsifiers, aromatizing products, binders, colorants, odorants, thickening agents, and the like.

In some embodiments, the microbial products of the present disclosure are solid. Where solid compositions are used, it may be desired to include one or more carrier materials including, but not limited to: mineral earths such as silicas, talc, kaolin, limestone, chalk, clay, dolomite, diatomaceous earth; calcium carbonate; calcium sulfate; magnesium sulfate; magnesium oxide; products of vegetable origin such as cereal meals, tree bark meal, wood meal, and nutshell meal.

In some embodiments, the microbial products of the present disclosure are liquid. In further embodiments, the liquid comprises a solvent that may include water or an alcohol, and other animal-safe solvents. In some embodiments, the microbial products of the present disclosure include binders such as animal-safe polymers, carboxymethylcellulose, starch, polyvinyl alcohol, and the like.

In some embodiments, the microbial products of the present disclosure comprise thickening agents such as silica, clay, natural extracts of seeds or seaweed, synthetic derivatives of cellulose, guar gum, locust bean gum, alginates, and methylcelluloses. In some embodiments, the microbial products comprise anti-settling agents such as modified starches, polyvinyl alcohol, xanthan gum, and the like.

In some embodiments, the microbial products of the present disclosure comprise colorants including organic chromophores classified as nitroso; nitro; azo, including monoazo, bisazo and polyazo; acridine, anthraquinone, azine, diphenylmethane, indamine, indophenol, methine, oxazine, phthalocyanine, thiazine, thiazole, triarylmethane, xanthene. In some embodiments, the microbial compositions of the present disclosure comprise trace nutrients such as salts of iron, manganese, boron, copper, cobalt, molybdenum, and zinc.

In some embodiments, the microbial products of the present disclosure comprise an animal-safe virucide or nematicide. In some embodiments, microbial compositions of the present disclosure comprise saccharides (e.g., monosaccharides, disaccharides, trisaccharides, polysaccharides, oligosaccharides, and the like), polymeric saccharides, lipids, polymeric lipids, lipopolysaccharides, proteins, polymeric proteins, lipoproteins, nucleic acids, nucleic acid polymers, silica, inorganic salts, and combinations thereof. In a further embodiment, microbial products comprise polymers of agar, agarose, gelrite, gellan gum and the like. In some embodiments, microbial compositions comprise plastic capsules, emulsions (e.g., water and oil), membranes, and artificial membranes. In some embodiments, emulsions or linked polymer solutions may comprise microbial compositions of the present disclosure. See, e.g., Harel and Bennett U.S. Pat. No. 8,460,726B2, the entirety of which is herein explicitly incorporated by reference for all purposes.

In some embodiments, the microbial products of the present disclosure comprise one or more preservatives. The preservatives may be in liquid or gas formulations. The preservatives may be selected from one or more of monosaccharide, disaccharide, trisaccharide, polysaccharide, acetic acid, ascorbic acid, calcium ascorbate, erythorbic acid, iso-ascorbic acid, erythrobic acid, potassium nitrate, sodium ascorbate, sodium erythorbate, sodium iso-ascorbate, sodium nitrate, sodium nitrite, nitrogen, benzoic acid, calcium sorbate, ethyl lauroyl arginate, methyl-p-hydroxy benzoate, methyl paraben, potassium acetate, potassium benzoiate, potassium bisulphite, potassium diacetate, potassium lactate, potassium metabisulphite, potassium sorbate, propyl-p-hydroxy benzoate, propyl paraben, sodium acetate, sodium benzoate, sodium bisulphite, sodium nitrite, sodium diacetate, sodium lactate, sodium metabisulphite, sodium salt of methyl-p-hydroxy benzoic acid, sodium salt of propyl-p-hydroxy benzoic acid, sodium sulphate, sodium sulfite, sodium dithionite, sulphurous acid, calcium propionate, dimethyl dicarbonate, natamycin, potassium sorbate, potassium bisulfite, potassium metabisulfite, propionic acid, sodium diacetate, sodium propionate, sodium sorbate, sorbic acid, ascorbic acid, ascorbyl palmitate, ascorbyl stearate, butylated hydro-xyanisole, butylated hydroxytoluene (BHT), butylated hydroxyl anisole (BHA), citric acid, citric acid esters of mono- and/or diglycerides, L-cysteine, L-cysteine hydrochloride, gum guaiacum, gum guaiac, lecithin, lecithin citrate, monoglyceride citrate, monoisopropyl citrate, propyl gallate, sodium metabisulphite, tartaric acid, tertiary butyl hydroquinone, stannous chloride, thiodipropionic acid, dilauryl thiodipropionate, distearyl thiodipropionate, ethoxyquin, sulfur dioxide, formic acid, or tocopherol(s).

In some embodiments, microbial products of the present disclosure include bacterial and/or fungal cells in spore form, vegetative cell form, and/or lysed cell form. In one embodiment, the lysed cell form acts as a mycotoxin binder, e.g. mycotoxins binding to dead cells.

In some embodiments, the microbial products are shelf stable in a refrigerator (35-40° F.) for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial products are shelf stable in a refrigerator (35-40° F.) for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some embodiments, the microbial products are shelf stable at room temperature (68-72° F.) or between 50-77° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial products are shelf stable at room temperature (68-72° F.) or between 50-77° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some embodiments, the microbial products are shelf stable at −23-35° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial products are shelf stable at −23-35° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some embodiments, the microbial products are shelf stable at 77-100° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial products are shelf stable at 77-100° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some embodiments, the microbial products are shelf stable at 101-213° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial products are shelf stable at 101-213° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some embodiments, the microbial products of the present disclosure are shelf stable at refrigeration temperatures (35-40° F.), at room temperature (68-72° F.), between 50-77° F., between −23-35° F., between 70-100° F., or between 101-213° F. for a period of about 1 to 100, about 1 to 95, about 1 to 90, about 1 to 85, about 1 to 80, about 1 to 75, about 1 to 70, about 1 to 65, about 1 to 60, about 1 to 55, about 1 to 50, about 1 to 45, about 1 to 40, about 1 to 35, about 1 to 30, about 1 to 25, about 1 to 20, about 1 to 15, about 1 to 10, about 1 to 5, about 5 to 100, about 5 to 95, about 5 to 90, about 5 to 85, about 5 to 80, about 5 to 75, about 5 to 70, about 5 to 65, about 5 to 60, about 5 to 55, about 5 to 50, about 5 to 45, about 5 to 40, about 5 to 35, about 5 to 30, about 5 to 25, about 5 to 20, about 5 to 15, about 5 to 10, about 10 to 100, about 10 to 95, about 10 to 90, about 10 to 85, about 10 to 80, about 10 to 75, about 10 to 70, about 10 to 65, about 10 to 60, about 10 to 55, about 10 to 50, about 10 to 45, about 10 to 40, about 10 to 35, about 10 to 30, about 10 to 25, about 10 to 20, about 10 to 15, about 15 to 100, about 15 to 95, about 15 to 90, about 15 to 85, about 15 to 80, about 15 to 75, about 15 to 70, about 15 to 65, about 15 to 60, about 15 to 55, about 15 to 50, about 15 to 45, about 15 to 40, about 15 to 35, about 15 to 30, about 15 to 25, about 15 to 20, about 20 to 100, about 20 to 95, about 20 to 90, about 20 to 85, about 20 to 80, about 20 to 75, about 20 to 70, about 20 to 65, about 20 to 60, about 20 to 55, about 20 to 50, about 20 to 45, about 20 to 40, about 20 to 35, about 20 to 30, about 20 to 25, about 25 to 100, about 25 to 95, about 25 to 90, about 25 to 85, about 25 to 80, about 25 to 75, about 25 to 70, about 25 to 65, about 25 to 60, about 25 to 55, about 25 to 50, about 25 to 45, about 25 to 40, about 25 to 35, about 25 to 30, about 30 to 100, about 30 to 95, about 30 to 90, about 30 to 85, about 30 to 80, about 30 to 75, about 30 to 70, about 30 to 65, about 30 to 60, about 30 to 55, about 30 to 50, about 30 to 45, about 30 to 40, about 30 to 35, about 35 to 100, about 35 to 95, about 35 to 90, about 35 to 85, about 35 to 80, about 35 to 75, about 35 to 70, about 35 to 65, about 35 to 60, about 35 to 55, about 35 to 50, about 35 to 45, about 35 to 40, about 40 to 100, about 40 to 95, about 40 to 90, about 40 to 85, about 40 to 80, about 40 to 75, about 40 to 70, about 40 to 65, about 40 to 60, about 40 to 55, about 40 to 50, about 40 to 45, about 45 to 100, about 45 to 95, about 45 to 90, about 45 to 85, about 45 to 80, about 45 to 75, about 45 to 70, about 45 to 65, about 45 to 60, about 45 to 55, about 45 to 50, about 50 to 100, about 50 to 95, about 50 to 90, about 50 to 85, about 50 to 80, about 50 to 75, about 50 to 70, about 50 to 65, about 50 to 60, about 50 to 55, about 55 to 100, about 55 to 95, about 55 to 90, about 55 to 85, about 55 to 80, about 55 to 75, about 55 to 70, about 55 to 65, about 55 to 60, about 60 to 100, about 60 to 95, about 60 to 90, about 60 to 85, about 60 to 80, about 60 to 75, about 60 to 70, about 60 to 65, about 65 to 100, about 65 to 95, about 65 to 90, about 65 to 85, about 65 to 80, about 65 to 75, about 65 to 70, about 70 to 100, about 70 to 95, about 70 to 90, about 70 to 85, about 70 to 80, about 70 to 75, about 75 to 100, about 75 to 95, about 75 to 90, about 75 to 85, about 75 to 80, about 80 to 100, about 80 to 95, about 80 to 90, about 80 to 85, about 85 to 100, about 85 to 95, about 85 to 90, about 90 to 100, about 90 to 95, or 95 to 100 weeks

In some embodiments, the microbial products of the present disclosure are shelf stable at refrigeration temperatures (35-40° F.), at room temperature (68-72° F.), between 50-77° F., between −23-35° F., between 70-100° F., or between 101-213° F. for a period of 1 to 100, 1 to 95, 1 to 90, 1 to 85, 1 to 80, 1 to 75, 1 to 70, 1 to 65, 1 to 60, 1 to 55, 1 to 50, 1 to 45, 1 to 40, 1 to 35, 1 to 30, 1 to 25, 1 to 20, 1 to 15, 1 to 10, 1 to 5, 5 to 100, 5 to 95, 5 to 90, 5 to 85, 5 to 80, 5 to 75, 5 to 70, 5 to 65, 5 to 60, 5 to 55, 5 to 50, 5 to 45, 5 to 40, 5 to 35, 5 to 30, 5 to 25, 5 to 20, 5 to 15, 5 to 10, 10 to 100, 10 to 95, 10 to 90, 10 to 85, 10 to 80, 10 to 75, 10 to 70, 10 to 65, 10 to 60, 10 to 55, 10 to 50, 10 to 45, 10 to 40, 10 to 35, 10 to 30, 10 to 25, 10 to 20, 10 to 15, 15 to 100, 15 to 95, 15 to 90, 15 to 85, 15 to 80, 15 to 75, 15 to 70, 15 to 65, 15 to 60, 15 to 55, 15 to 50, 15 to 45, 15 to 40, 15 to 35, 15 to 30, 15 to 25, 15 to 20, 20 to 100, 20 to 95, 20 to 90, 20 to 85, 20 to 80, 20 to 75, 20 to 70, 20 to 65, 20 to 60, 20 to 55, 20 to 50, 20 to 45, 20 to 40, 20 to 35, 20 to 30, 20 to 25, 25 to 100, 25 to 95, 25 to 90, 25 to 85, 25 to 80, 25 to 75, 25 to 70, 25 to 65, 25 to 60, 25 to 55, 25 to 50, 25 to 45, 25 to 40, 25 to 35, 25 to 30, 30 to 100, 30 to 95, 30 to 90, 30 to 85, 30 to 80, 30 to 75, 30 to 70, 30 to 65, 30 to 60, 30 to 55, 30 to 50, 30 to 45, 30 to 40, 30 to 35, 35 to 100, 35 to 95, 35 to 90, 35 to 85, 35 to 80, 35 to 75, 35 to 70, 35 to 65, 35 to 60, 35 to 55, 35 to 50, 35 to 45, 35 to 40, 40 to 100, 40 to 95, 40 to 90, 40 to 85, 40 to 80, 40 to 75, 40 to 70, 40 to 65, 40 to 60, 40 to 55, 40 to 50, 40 to 45, 45 to 100, 45 to 95, 45 to 90, 45 to 85, 45 to 80, 45 to 75, 45 to 70, 45 to 65, 45 to 60, 45 to 55, 45 to 50, 50 to 100, 50 to 95, 50 to 90, 50 to 85, 50 to 80, 50 to 75, 50 to 70, 50 to 65, 50 to 60, 50 to 55, 55 to 100, 55 to 95, 55 to 90, 55 to 85, 55 to 80, 55 to 75, 55 to 70, 55 to 65, 55 to 60, 60 to 100, 60 to 95, 60 to 90, 60 to 85, 60 to 80, 60 to 75, 60 to 70, 60 to 65, 65 to 100, 65 to 95, 65 to 90, 65 to 85, 65 to 80, 65 to 75, 65 to 70, 70 to 100, 70 to 95, 70 to 90, 70 to 85, 70 to 80, 70 to 75, 75 to 100, 75 to 95, 75 to 90, 75 to 85, 75 to 80, 80 to 100, 80 to 95, 80 to 90, 80 to 85, 85 to 100, 85 to 95, 85 to 90, 90 to 100, 90 to 95, or 95 to 100 weeks.

In some embodiments, the microbial products of the present disclosure are shelf stable at refrigeration temperatures (35-40° F.), at room temperature (68-72° F.), between 50-77° F., between −23-35° F., between 70-100° F., or between 101-213° F. for a period of about 1 to 36, about 1 to 34, about 1 to 32, about 1 to 30, about 1 to 28, about 1 to 26, about 1 to 24, about 1 to 22, about 1 to 20, about 1 to 18, about 1 to 16, about 1 to 14, about 1 to 12, about 1 to 10, about 1 to 8, about 1 to 6, about 1 one 4, about 1 to 2, about 4 to 36, about 4 to 34, about 4 to 32, about 4 to 30, about 4 to 28, about 4 to 26, about 4 to 24, about 4 to 22, about 4 to 20, about 4 to 18, about 4 to 16, about 4 to 14, about 4 to 12, about 4 to 10, about 4 to 8, about 4 to 6, about 6 to 36, about 6 to 34, about 6 to 32, about 6 to 30, about 6 to 28, about 6 to 26, about 6 to 24, about 6 to 22, about 6 to 20, about 6 to 18, about 6 to 16, about 6 to 14, about 6 to 12, about 6 to 10, about 6 to 8, about 8 to 36, about 8 to 34, about 8 to 32, about 8 to 30, about 8 to 28, about 8 to 26, about 8 to 24, about 8 to 22, about 8 to 20, about 8 to 18, about 8 to 16, about 8 to 14, about 8 to 12, about 8 to 10, about 10 to 36, about 10 to 34, about 10 to 32, about 10 to 30, about 10 to 28, about 10 to 26, about 10 to 24, about 10 to 22, about 10 to 20, about 10 to 18, about 10 to 16, about 10 to 14, about 10 to 12, about 12 to 36, about 12 to 34, about 12 to 32, about 12 to 30, about 12 to 28, about 12 to 26, about 12 to 24, about 12 to 22, about 12 to 20, about 12 to 18, about 12 to 16, about 12 to 14, about 14 to 36, about 14 to 34, about 14 to 32, about 14 to 30, about 14 to 28, about 14 to 26, about 14 to 24, about 14 to 22, about 14 to 20, about 14 to 18, about 14 to 16, about 16 to 36, about 16 to 34, about 16 to 32, about 16 to 30, about 16 to 28, about 16 to 26, about 16 to 24, about 16 to 22, about 16 to 20, about 16 to 18, about 18 to 36, about 18 to 34, about 18 to 32, about 18 to 30, about 18 to 28, about 18 to 26, about 18 to 24, about 18 to 22, about 18 to 20, about 20 to 36, about 20 to 34, about 20 to 32, about 20 to 30, about 20 to 28, about 20 to 26, about 20 to 24, about 20 to 22, about 22 to 36, about 22 to 34, about 22 to 32, about 22 to 30, about 22 to 28, about 22 to 26, about 22 to 24, about 24 to 36, about 24 to 34, about 24 to 32, about 24 to 30, about 24 to 28, about 24 to 26, about 26 to 36, about 26 to 34, about 26 to 32, about 26 to 30, about 26 to 28, about 28 to 36, about 28 to 34, about 28 to 32, about 28 to 30, about 30 to 36, about 30 to 34, about 30 to 32, about 32 to 36, about 32 to 34, or about 34 to 36 months.

In some embodiments, the microbial products of the present disclosure are shelf stable at refrigeration temperatures (35-40° F.), at room temperature (68-72° F.), between 50-77° F., between −23-35° F., between 70-100° F., or between 101-213° F. for a period of 1 to 36 1 to 34 1 to 32 1 to 30 1 to 28 1 to 26 1 to 24 1 to 22 1 to 20 1 to 18 1 to 16 1 to 14 1 to 12 1 to 10 1 to 8 1 to 6 1 one 4 1 to 2 4 to 36 4 to 34 4 to 32 4 to 30 4 to 28 4 to 26 4 to 24 4 to 22 4 to 20 4 to 18 4 to 16 4 to 14 4 to 12 4 to 10 4 to 8 4 to 6 6 to 36 6 to 34 6 to 32 6 to 30 6 to 28 6 to 26 6 to 24 6 to 22 6 to 20 6 to 18 6 to 16 6 to 14 6 to 12 6 to 10 6 to 8 8 to 36 8 to 34 8 to 32 8 to 30 8 to 28 8 to 26 8 to 24 8 to 22 8 to 20 8 to 18 8 to 16 8 to 14 8 to 12 8 to 10 10 to 36 10 to 34 10 to 32 10 to 30 10 to 28 10 to 26 10 to 24 10 to 22 10 to 20 10 to 18 10 to 16 10 to 14 10 to 12 12 to 36 12 to 34 12 to 32 12 to 30 12 to 28 12 to 26 12 to 24 12 to 22 12 to 20 12 to 18 12 to 16 12 to 14 14 to 36 14 to 34 14 to 32 14 to 30 14 to 28 14 to 26 14 to 24 14 to 22 14 to 20 14 to 18 14 to 16 16 to 36 16 to 34 16 to 32 16 to 30 16 to 28 16 to 26 16 to 24 16 to 22 16 to 20 16 to 18 18 to 36 18 to 34 18 to 32 18 to 30 18 to 28 18 to 26 18 to 24 18 to 22 18 to 20 20 to 36 20 to 34 20 to 32 20 to 30 20 to 28 20 to 26 20 to 24 20 to 22 22 to 36 22 to 34 22 to 32 22 to 30 22 to 28 22 to 26 22 to 24 24 to 36 24 to 34 24 to 32 24 to 30 24 to 28 24 to 26 26 to 36 26 to 34 26 to 32 26 to 30 26 to 28 28 to 36 28 to 34 28 to 32 28 to 30 30 to 36 30 to 34 30 to 32 32 to 36 32 to 34, or about 34 to 36.

In some embodiments, the microbial products of the present disclosure are shelf stable at any of the disclosed temperatures and/or temperature ranges and spans of time at a relative humidity of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, or 98%

### Encapsulated Products

In some embodiments, the viability-enhanced microbe(s) (e.g., the microbes and/or synthetic microbial compositions) of the disclosure are encapsulated in an encapsulating composition. An encapsulating composition protects the microbes from external stressors prior to entering the gastrointestinal tract of ungulates. Encapsulating compositions further create an environment that may be beneficial to the microbes, such as minimizing the oxidative stresses of an aerobic environment on anaerobic microbes. See Kalsta et al. (U.S. Pat. No. 5,104,662A), Ford (U.S. Pat. No. 5,733,568A), and Mosbach and Nilsson (U.S. Pat. No. 4,647,536A) for encapsulation compositions of microbes, and methods of encapsulating microbes. Additional method and formulations of synthetic ensembles can include formulations and methods as disclosed in one or more of the following U.S. Pat. Nos. 6,537,666, 6,306,345, 5,766,520, 6,509,146, 6,884,866, 7,153,472, 6,692,695, 6,872,357, 7,074,431, and/or 6534087, each of which is herein expressly incorporated by reference in its entirety.

In one embodiment, the encapsulating composition comprises microcapsules having a multiplicity of liquid cores encapsulated in a solid shell material. For purposes of the disclosure, a “multiplicity” of cores is defined as two or more.

A first category of useful fusible shell materials is that of normally solid fats, including fats which are already of suitable hardness and animal or vegetable fats and oils which are hydrogenated until their melting points are sufficiently high to serve the purposes of the present disclosure. Depending on the desired process and storage temperatures and the specific material selected, a particular fat can be either a normally solid or normally liquid material. The terms “normally solid” and “normally liquid” as used herein refer to the state of a material at desired temperatures for storing the resulting microcapsules. Since fats and hydrogenated oils do not, strictly speaking, have melting points, the term “melting point” is used herein to describe the minimum temperature at which the fusible material becomes sufficiently softened or liquid to be successfully emulsified and spray cooled, thus roughly corresponding to the maximum temperature at which the shell material has sufficient integrity to prevent release of the choline cores. “Melting point” is similarly defined herein for other materials which do not have a sharp melting point.

Specific examples of fats and oils useful herein (some of which require hardening) are as follows: animal oils and fats, such as beef tallow, mutton tallow, lamb tallow, lard or pork fat, fish oil, and sperm oil; vegetable oils, such as canola oil, cottonseed oil, peanut oil, corn oil, olive oil, soybean oil, sunflower oil, safflower oil, coconut oil, palm oil, linseed oil, tung oil, and castor oil; fatty acid monoglycerides and diglycerides; free fatty acids, such as stearic acid, palmitic acid, and oleic acid; and mixtures thereof. The above listing of oils and fats is not meant to be exhaustive, but only exemplary. Specific examples of fatty acids include linoleic acid, γ-linoleic acid, dihomo-γ-linolenic acid, arachidonic acid, docosatetraenoic acid, vaccenic acid, nervonic acid, mead acid, erucic acid, gondoic acid, elaidic acid, oleic acid, palitoleic acid, stearidonic acid, eicosapentaenoic acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecyclic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, heptatriacontanoic acid, and octatriacontanoic acid.

Another category of fusible materials useful as encapsulating shell materials is that of waxes. Representative waxes contemplated for use herein are as follows: animal waxes, such as beeswax, lanolin, shell wax, and Chinese insect wax; vegetable waxes, such as carnauba, candelilla, bayberry, and sugar cane; mineral waxes, such as paraffin, microcrystalline petroleum, ozocerite, ceresin, and montan; synthetic waxes, such as low molecular weight polyolefin (e.g., CARBOWAX), and polyol ether-esters (e.g., sorbitol); Fischer-Tropsch process synthetic waxes; and mixtures thereof. Water-soluble waxes, such as CARBOWAX and sorbitol, are not contemplated herein if the core is aqueous.

Still other fusible compounds useful herein are fusible natural resins, such as rosin, balsam, shellac, and mixtures thereof. Various adjunct materials are contemplated for incorporation in fusible materials according to the present disclosure. For example, antioxidants, light stabilizers, dyes and lakes, flavors, essential oils, anti-caking agents, fillers, pH stabilizers, sugars (monosaccharides, disaccharides, trisaccharides, and polysaccharides) and the like can be incorporated in the fusible material in amounts which do not diminish its utility for the present disclosure. The core material contemplated according to some embodiments herein constitutes from about 0.1% to about 50%, about 1% to about 35%, or about 5% to about 30% by weight of the microcapsules. In some embodiments, the core material contemplated herein constitutes no more than about 30% by weight of the microcapsules. In some embodiments, the core material contemplated herein constitutes about 5% by weight of the microcapsules. Depending on the implementation, the core material can be a liquid or solid at contemplated storage temperatures of the microcapsules.

The cores can include other additives, including edible sugars, such as sucrose, glucose, maltose, fructose, lactose, cellobiose, monosaccharides, disaccharides, trisaccharides, polysaccharides, and mixtures thereof; artificial sweeteners, such as aspartame, saccharin, cyclamate salts, and mixtures thereof; edible acids, such as acetic acid (vinegar), citric acid, ascorbic acid, tartaric acid, and mixtures thereof; edible starches, such as corn starch; hydrolyzed vegetable protein; water-soluble vitamins, such as Vitamin C; water-soluble medicaments; water-soluble nutritional materials, such as ferrous sulfate; flavors; salts; monosodium glutamate; antimicrobial agents, such as sorbic acid; antimycotic agents, such as potassium sorbate, sorbic acid, sodium benzoate, and benzoic acid; food grade pigments and dyes; and mixtures thereof. Other potentially useful supplemental core materials are also contemplated, depending on the implementation.

Emulsifying agents can be utilized in some embodiments to assist in the formation of stable emulsions. Representative emulsifying agents include glyceryl monostearate, polysorbate esters, ethoxylated mono- and diglycerides, and mixtures thereof.

For ease of processing, and particularly to enable the successful formation of a reasonably stable emulsion, the viscosities of the core material and the shell material should be similar at the temperature at which the emulsion is formed. In some embodiments, the ratio of the viscosity of the shell to the viscosity of the core, expressed in centipoise or comparable units, and both measured at the temperature of the emulsion, can be from about 22:1 to about 1:1, from about 8:1 to about 1:1, or from about 3:1 to about 1:1. A ratio of 1:1 can be utilized in some embodiments, and other viscosities can be employed for various applications where a viscosity ratio within the recited ranges is useful.

Encapsulating compositions are not limited to microcapsule compositions as disclosed above. In some embodiments encapsulating compositions encapsulate the microbial compositions in an adhesive polymer that can be natural or synthetic without toxic effect. In some embodiments, the encapsulating composition may be a matrix selected from sugar matrix, gelatin matrix, polymer matrix, silica matrix, starch matrix, foam matrix, etc. In some embodiments, the encapsulating composition may be selected from polyvinyl acetates; polyvinyl acetate copolymers; ethylene vinyl acetate (EVA) copolymers; polyvinyl alcohols; polyvinyl alcohol copolymers; celluloses, including ethylcelluloses, methylcelluloses, hydroxymethylcelluloses, hydroxypropylcelluloses and carboxymethylcellulose; polyvinylpyrolidones; polysaccharides, including starch, modified starch, dextrins, maltodextrins, alginate and chitosans; monosaccharides; fats; fatty acids, including oils; proteins, including gelatin and zeins; gum arabics; shellacs; vinylidene chloride and vinylidene chloride copolymers; calcium lignosulfonates; acrylic copolymers; polyvinylacrylates; polyethylene oxide: acrylamide polymers and copolymers; polyhydroxyethyl acrylate, methylacrylamide monomers; and polychloroprene.

In some embodiments, the encapsulating shell of the present disclosure can be up to 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 510 μm, 520 μm, 530 μm, 540 μm, 550 μm, 560 μm, 570 μm, 580 μm, 590 μm, 600 μm, 610 μm, 620 μm, 630 μm, 640 μm, 650 μm, 660 μm, 670 μm, 680 μm, 690 μm, 700 μm, 710 μm, 720 μm, 730 μm, 740 μm, 750 μm, 760 μm, 770 μm, 780 μm, 790 μm, 800 μm, 810 μm, 820 μm, 830 μm, 840 μm, 850 μm, 860 μm, 870 μm, 880 μm, 890 μm, 900 μm, 910 μm, 920 μm, 930 μm, 940 μm, 950 μm, 960 μm, 970 μm, 980 μm, 990 μm, 1000 μm, 1010 μm, 1020 μm, 1030 μm, 1040 μm, 1050 μm, 1060 μm, 1070 μm, 1080 μm, 1090 μm, 1100 μm, 1110 μm, 1120 μm, 1130 μm, 1140 μm, 1150 μm, 1160 μm, 1170 μm, 1180 μm, 1190 μm, 1200 μm, 1210 μm, 1220 μm, 1230 μm, 1240 μm, 1250 μm, 1260 μm, 1270 μm, 1280 μm, 1290 μm, 1300 μm, 1310 μm, 1320 μm, 1330 μm, 1340 μm, 1350 μm, 1360 μm, 1370 μm, 1380 μm, 1390 μm, 1400 μm, 1410 μm, 1420 μm, 1430 μm, 1440 μm, 1450 μm, 1460 μm, 1470 μm, 1480 μm, 1490 μm, 1500 μm, 1510 μm, 1520 μm, 1530 μm, 1540 μm, 1550 μm, 1560 μm, 1570 μm, 1580 μm, 1590 μm, 1600 μm, 1610 μm, 1620 μm, 1630 μm, 1640 μm, 1650 μm, 1660 μm, 1670 μm, 1680 μm, 1690 μm, 1700 μm, 1710 μm, 1720 μm, 1730 μm, 1740 μm, 1750 μm, 1760 μm, 1770 μm, 1780 μm, 1790 μm, 1800 μm, 1810 μm, 1820 μm, 1830 μm, 1840 μm, 1850 μm, 1860 μm, 1870 μm, 1880 μm, 1890 μm, 1900 μm, 1910 μm, 1920 μm, 1930 μm, 1940 μm, 1950 μm, 1960 μm, 1970 μm, 1980 μm, 1990 μm, 2000 μm, 2010 μm, 2020 μm, 2030 μm, 2040 μm, 2050 μm, 2060 μm, 2070 μm, 2080 μm, 2090 μm, 2100 μm, 2110 μm, 2120 μm, 2130 μm, 2140 μm, 2150 μm, 2160 μm, 2170 μm, 2180 μm, 2190 μm, 2200 μm, 2210 μm, 2220 μm, 2230 μm, 2240 μm, 2250 μm, 2260 μm, 2270 μm, 2280 μm, 2290 μm, 2300 μm, 2310 μm, 2320 μm, 2330 μm, 2340 μm, 2350 μm, 2360 μm, 2370 μm, 2380 μm, 2390 μm, 2400 μm, 2410 μm, 2420 μm, 2430 μm, 2440 μm, 2450 μm, 2460 μm, 2470 μm, 2480 μm, 2490 μm, 2500 μm, 2510 μm, 2520 μm, 2530 μm, 2540 μm, 2550 μm, 2560 μm, 2570 μm, 2580 μm, 2590 μm, 2600 μm, 2610 μm, 2620 μm, 2630 μm, 2640 μm, 2650 μm, 2660 μm, 2670 μm, 2680 μm, 2690 μm, 2700 μm, 2710 μm, 2720 μm, 2730 μm, 2740 μm, 2750 μm, 2760 μm, 2770 μm, 2780 μm, 2790 μm, 2800 μm, 2810 μm, 2820 μm, 2830 μm, 2840 μm, 2850 μm, 2860 μm, 2870 μm, 2880 μm, 2890 μm, 2900 μm, 2910 μm, 2920 μm, 2930 μm, 2940 μm, 2950 μm, 2960 μm, 2970 μm, 2980 μm, 2990 μm, or 3000 μm thick.

### Animal Feed

In some embodiments, the microbial products of the present disclosure are mixed with animal feed. In some embodiments, animal feed may be present in various forms such as pellets, capsules, granulated, powdered, liquid, or semi-liquid.

In some embodiments, products of the present disclosure are mixed into the premix at the feed mill (e.g., Cargill or Western Millin), alone as a standalone premix, and/or alongside other feed additives such as MONENSIN, vitamins, etc. In one embodiment, the products of the present disclosure are mixed into the feed at the feed mill. In another embodiment, products of the present disclosure are mixed into the feed itself.

In some embodiments, the feed may be supplemented with water, premix or premixes, forage, fodder, beans (e.g., whole, cracked, or ground), grains (e.g., whole, cracked, or ground), bean- or grain-based oils, bean- or grain-based meals, bean- or grain-based haylage or silage, bean- or grain-based syrups, fatty acids, sugar alcohols (e.g., polyhydric alcohols), commercially available formula feeds, and mixtures thereof.

In some embodiments, forage encompasses hay, haylage, and silage. In some embodiments, hays include grass hays (e.g., sudangrass, orchardgrass, or the like), alfalfa hay, and clover hay. In some embodiments, haylages include grass haylages, sorghum haylage, and alfalfa haylage. In some embodiments, silages include maize, oat, wheat, alfalfa, clover, and the like.

In some embodiments, premix or premixes may be utilized in the feed. Premixes may comprise micro-ingredients such as vitamins, minerals, amino acids; chemical preservatives; pharmaceutical compositions such as antibiotics and other medicaments; fermentation products, and other ingredients. In some embodiments, premixes are blended into the feed.

In some embodiments, the feed may include feed concentrates such as soybean hulls, sugar beet pulp, molasses, high protein soybean meal, ground corn, shelled corn, wheat midds, distiller grain, cottonseed hulls, rumen-bypass protein, rumen-bypass fat, and grease. See Luhman (U.S. Publication US20150216817A1), Anderson et al. (U.S. Pat. No. 3,484,243) and Porter and Luhman (U.S. Pat. No. 9,179,694B2) for animal feed and animal feed supplements capable of use in the present compositions and methods.

In some embodiments, feed occurs as a compound, which includes, in a mixed composition capable of meeting the basic dietary needs, the feed itself, vitamins, minerals, amino acids, and other necessary components. Compound feed may further comprise premixes.

In some embodiments, microbial compositions of the present disclosure may be mixed with animal feed, premix, and/or compound feed. Individual components of the animal feed may be mixed with the microbial compositions prior to feeding to ruminants. The microbial compositions of the present disclosure may be applied into or on a premix, into or on a feed, and/or into or on a compound feed.

### Microbial Culture Techniques

The isolation, identification, and culturing of the microbes of the present disclosure can be effected using standard microbiological techniques. Examples of such techniques may be found in Gerhardt, P. (ed.) Methods for General and Molecular Microbiology. American Society for Microbiology, Washington, D.C. (1994) and Lennette, E. H. (ed.) Manual of Clinical Microbiology, Third Edition. American Society for Microbiology, Washington, D.C. (1980), each of which is incorporated by reference.

Isolation can be effected by streaking the specimen on a solid medium (e.g., nutrient agar plates) to obtain a single colony, which is characterized by the phenotypic traits described hereinabove (e.g., Gram positive/negative, capable of forming spores aerobically/anaerobically, cellular morphology, carbon source metabolism, acid/base production, enzyme secretion, metabolic secretions, etc.) and to reduce the likelihood of working with a culture which has become contaminated.

For example, for microbes of the disclosure, biologically pure isolates can be obtained through repeated subculture of biological samples, each subculture followed by streaking onto solid media to obtain individual colonies or colony forming units. Methods of preparing, thawing, and growing lyophilized bacteria are commonly known, for example, Gherna, R L. and C. A. Reddy. 2007. Culture Preservation, p 1019-1033. In C. A. Reddy, T. J. Beveridge, J. A. Breznak, G. A. Marzluf, T. M. Schmidt, and L. R. Snyder, eds. American Society for Microbiology, Washington, D.C., 1033 pages; herein incorporated by reference. Thus freeze dried liquid formulations and cultures stored long term at −70° C. in solutions containing glycerol are contemplated for use in providing formulations of the present disclosure.

The microbes of the disclosure can be propagated in a liquid medium under aerobic conditions, or alternatively anaerobic conditions. Medium for growing the bacterial strains of the present disclosure includes a carbon source, a nitrogen source, and inorganic salts, as well as specially required substances such as vitamins, amino acids, nucleic acids and the like. Examples of suitable carbon sources which can be used for growing the microbes include, but are not limited to, starch, peptone, yeast extract, amino acids, sugars such as glucose, arabinose, mannose, glucosamine, maltose, and the like; salts of organic acids such as acetic acid, fumaric acid, adipic acid, propionic acid, citric acid, gluconic acid, malic acid, pyruvic acid, malonic acid and the like; alcohols such as ethanol and glycerol and the like; oil or fat such as soybean oil, rice bran oil, olive oil, corn oil, sesame oil. The amount of the carbon source added varies according to the kind of carbon source and is typically between 1 to 100 g/L. Preferably, glucose, starch, and/or peptone is contained in the medium as a major carbon source, at a concentration of 0.1-5% (W/V).

Examples of suitable nitrogen sources which can be used for growing the bacterial strains of the present disclosure include, but are not limited to, amino acids, yeast extract, tryptone, beef extract, peptone, potassium nitrate, ammonium nitrate, ammonium chloride, ammonium sulfate, ammonium phosphate, ammonia, or combinations thereof. The amount of nitrogen source varies according to the type of nitrogen source, typically between 0.1 g/L to 30 g/L.

The inorganic salts, potassium dihydrogen phosphate, dipotassium hydrogen phosphate, disodium hydrogen phosphate, magnesium sulfate, magnesium chloride, ferric sulfate, ferrous sulfate, ferric chloride, ferrous chloride, manganous sulfate, manganous chloride, zinc sulfate, zinc chloride, cupric sulfate, calcium chloride, sodium chloride, calcium carbonate, sodium carbonate can be used alone or in combination. The amount of inorganic acid varies according to the kind of the inorganic salt, typically between 0.001 g/L to 10 g/L. Examples of specially required substances include, but are not limited to, vitamins, nucleic acids, yeast extract, peptone, meat extract, malt extract, dried yeast, and combinations thereof.

Cultivation can be effected at a temperature, which allows the growth of the microbial strains, essentially, between 20° C. and 46° C. In some aspects, a temperature range is 30° C.-39° C. For optimal growth, in some embodiments, the medium can be adjusted to pH 6.0-7.4. It will be appreciated that commercially available media may also be used to culture the microbial strains, such as Nutrient Broth or Nutrient Agar available from Difco, Detroit, Mich. It will be appreciated that cultivation time may differ depending on the type of culture medium used and the concentration of sugar as a major carbon source.

In some aspects, cultivation lasts between 8-96 hours. Microbial cells thus obtained are isolated using methods which are well known in the art. Examples include, but are not limited to, membrane filtration and centrifugal separation. The pH may be adjusted using sodium hydroxide and the like and the culture may be dried using a freeze dryer, until the water content becomes equal to 4% or less. Microbial co-cultures may be obtained by propagating each strain as described herein above. In some aspects, microbial multi-strain cultures may be obtained by propagating two or more of the strains described hereinabove. It will be appreciated that the microbial strains may be cultured together when compatible culture conditions can be employed.

### FURTHER NUMBERED EMBODIMENTS

Further numbered embodiments of the present disclosure are provided as follows:

### Embodiment 1

A method of improving microbe viability after preservation comprising: subjecting a population of target microbial cells to a first preservation challenge to provide a population of challenged microbial cells; harvesting viable challenged microbial cells from the population of challenged microbial cells; preserving the viable challenged microbial cells to provide a population of preserved viability-enhanced microbial cells; and preparing a product using the population of preserved viability-enhanced microbial cells.

### Embodiment 2

The method of claim 1, wherein the first preservation challenge includes one of freeze drying, lyophilization, cryopreservation, preservation by evaporation, preservation by foam formation, vitrification, stabilization by glass formation, preservation by vaporization, spray drying, adsorptive drying, extrusion, or fluid bed drying.

### Embodiment 3

The method of claim 1 or claim 2, wherein preserving the viable challenged cells includes freeze drying, lyophilization, cryopreservation, preservation by evaporation, preservation by foam formation, vitrification, stabilization by glass formation, preservation by vaporization, spray drying, adsorptive drying, extrusion drying, or fluid bed drying.

### Embodiment 4

The method of any one of claims 1-3, further comprising subjecting the population of challenged cells to at least one additional preservation challenge

### Embodiment 5

A method for microbe viability enhancement and preservation, the method comprising: subjecting a population of target microbial cells to a first preservation challenge to provide a first population of challenged microbial cells; harvesting viable challenged microbial cells from the first population of challenged microbial cells to provide a first population of viable challenged microbial cells; subjecting the first population of viable challenged microbial cells to a second preservation challenge to provide a second population of challenged microbial cells; harvesting viable challenged microbial cells from the second population of challenged microbial cells to provide a second population of viable challenged microbial cells; preserving the second population of viable challenged microbial cells to provide a population of preserved viability-enhanced microbial cells; and preparing a product using the population of preserved viability-enhanced microbial cells.

### Embodiment 6

The method of claim 5, wherein the first preservation challenge and the second preservation challenge are of the same challenge type.

### Embodiment 7

The method of claim 5, wherein the first preservation challenge and the second preservation challenge are of different challenge types.

### Embodiment 8

The method of claim 5, wherein the first preservation challenge and the second preservation challenge are selected from a combination described in Table 1.

### Embodiment 9

The method of any one of claims 5-8, further comprising subjecting the second population of challenged cells to at least one additional preservation challenge.

### Embodiment 10

The method of any one of claims 5-9, wherein preserving the second viable challenged cell population includes freeze drying, lyophilization, cryopreservation, preservation by evaporation, preservation by foam formation, vitrification, stabilization by glass formation, preservation by vaporization, spray drying, adsorptive drying, extrusion drying, or fluid bed drying.

### Embodiment 11

The method of any one of claims 1-10, wherein the population of target microbial cells comprises a Clostridium spp. bacterium, a Succinivibrio spp. bacterium, a Butyrivibio spp. bacterium, a Bacillus spp. bacterium, a Lactobacillus spp. bacterium, a Prevotella spp. bacterium, a Syntrophococcus spp. bacterium, or a Ruminococcus spp. bacterium.

### Embodiment 12

The method of any one of claims 1-10, wherein the population of target microbial cells comprises a Caecomyces spp. fungus, a Pichia spp. fungus, an Orpinomyces spp. fungus, or a Piromyces spp. fungus.

### Embodiment 13

The method of any one of claims 1-10, wherein the population of target microbial cells comprises a species of the Lachnospiraceae family.

### Embodiment 14

The method of any one of claims 11-13, wherein: the Clostridium spp. comprises a 16S rRNA sequence comprising at least 97% sequence identity to SEQ ID NO: 1, SEQ ID NO: 3, SEQ ID NO: 5, or SEQ ID NO: 6; the Succinivibrio spp. comprises a 16S rRNA sequence comprising at least 97% sequence identity to SEQ ID NO: 11; the Pichia spp. comprises an ITS sequence comprising at least 97% sequence identity to SEQ ID NO: 2; the Bacillus spp. comprises a 16S rRNA sequence comprising at least 97% sequence identity to SEQ ID NO: 4; the Lactobacillus spp. comprises a 16S rRNA sequence comprising at least 97% sequence identity to SEQ ID NO: 7, SEQ ID NO: 8, or SEQ ID NO: 9; the Prevotella spp. comprises a 16S rRNA sequence comprising at least 97% sequence identity to SEQ ID NO: 10; or the species of the Lachnospiraceae family comprises a 16S rRNA sequence comprising at least 97% sequence identity to SEQ ID NO: 12.

### Embodiment 15

The method of any one of claims 1-10, wherein the population of target microbial cells comprises a Ruminococcus bovis bacterium, a Succinivibrio dextrinosolvens bacterium, or a Caecomyces spp. fungus.

### Embodiment 16

The method of any one of claims 1-10, wherein the population of target microbial cells comprises a Clostridium butyricum bacterium, a Pichia kudriazevii fungus, a Butyrivibio fibrosolvens bacterium, a Ruminococcus bovis bacterium, or a Succinivibrio dextrinosolvens bacterium.

### Embodiment 17

A product prepared by the methods of any one of claims 1-16, comprising a population of preserved viability-enhanced microbial cells.

### Embodiment 18

The product of claim 17, wherein the population of preserved viability-enhanced microbial cells comprises a Clostridium spp. bacterium, a Succinivibrio spp. bacterium, a Caecomyces spp. fungus, a Pichia spp. fungus, a Butyrivibio spp. bacterium, an Orpinomyces spp. fungus, a Piromyces spp. fungus, a Bacillus spp. bacterium, a Lactobacillus spp. bacterium, a Prevotella spp. bacterium, a Syntrophococcus spp. bacterium, a Ruminococcus spp bacterium, or a species of the Lachnospiraceae family.

### Embodiment 19

The product of claim 18, wherein: the Clostridium spp. comprises a 16S rRNA sequence comprising at least 97% sequence identity to SEQ ID NO: 1, SEQ ID NO: 3, SEQ ID NO: 5, or SEQ ID NO: 6; the Succinivibrio spp. comprises a 16S rRNA sequence comprising at least 97% sequence identity to SEQ ID NO: 11; the Pichia spp. comprises an ITS sequence comprising at least 97% sequence identity to SEQ ID NO: 2; the Bacillus spp. comprises a 16S rRNA sequence comprising at least 97% sequence identity to SEQ ID NO: 4; the Lactobacillus spp. comprises a 16S rRNA sequence comprising at least 97% sequence identity to SEQ ID NO: 7, SEQ ID NO: 8, or SEQ ID NO: 9; the Prevotella spp. comprises a 16S rRNA sequence comprising at least 97% sequence identity to SEQ ID NO: 10; or the species of the Lachnospiraceae family comprises a 16S rRNA sequence comprising at least 97% sequence identity to SEQ ID NO: 12.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### EXAMPLES

The present disclosure is further illustrated by reference to the following Experimental Data and Examples. However, it should be noted that these Experimental Data and Examples, like the embodiments described above, are illustrative and are not to be construed as restricting the scope of the disclosure in any way.

### Example 1—Preservation by Vaporization Challenge and Recovery Protocol

The following protocol describes the methods and regents for serial application of preservation by vaporization (PBV) to produce preserved bacteria compositions.

First, an aliquot from a research cell bank (RBC) glycerol stock is streaked onto a growth plate. After an appropriate incubation time, a single colony is selected and used to inoculate a seed tube of Tryptic Soy Broth. The seed tube inoculate is cultured to allow bacterial expansion and the expanded bacterial culture is then used to inoculate the main fermentation culture. The bacterial cells are cultured in the main fermentation culture until mid-stationary phase. Loading sugars are included, if necessary, at 5% w/v. After 40 hours, cells are harvested and combined with preservation solutions to produce a preservation mixture. Exemplary preservation solutions are provided below in Tables 3A-3C. Each strain was diluted tenfold in preservation mixture (100 μL of culture with 900 μL of Preservation solution).

TABLE 3A
Exemplary Preservation Solution

| Ingredient | g/L |
|---|---|
| d.i. water | 500 |
| Sorbitol | 50 |
| Monosodium Glutamate | 100 |
| Sucrose | 150 |
| Polyvinyl-pyrrolidone K-15 | 50 |
| Potassium Phosphate, Monobasic | 0.354 |
| Potassium Phosphate, Dibasic | 1.27 |
| 0.1% resazurin | 2.00 mL |

pH adjusted to 7.0 ± 0.05 with 1-5 M NaOH of HCl
Q.S. to 1 liter with a graduated cylinder

TABLE 3B
Exemplary Preservation Solution

| Ingredient | g/L |
|---|---|
| d.i. water | 500 |
| Mannitol | 50 |
| Monosodium Glutamate | 100 |
| Polyethylene glycol | 150 |
| Polyvinyl-pyrrolidone K-15 | 50 |
| Potassium Phosphate, Monobasic | 0.354 |
| Potassium Phosphate, Dibasic | 1.27 |
| 0.1% resazurin | 2.00 mL |

pH adjusted to 7.0 ± 0.05 with 1-5 M NaOH of HCl
Q.S. to 1 liter with a graduated cylinder

TABLE 3C
Exemplary Preservation Solution

| Ingredient | g/L |
|---|---|
| d.i. water | 500 |
| Glycerol | 50 |
| Monosodium Glutamate | 100 |
| Trehalose | 150 |
| Polyvinyl-pyrrolidone K-15 | 50 |
| Potassium Phosphate, Monobasic | 0.354 |
| Potassium Phosphate, Dibasic | 1.27 |
| 0.1% resazurin | 2.00 mL |

pH adjusted to 7.0 ± 0.05 with 1-5 M NaOH of HCl
Q.S. to 1 liter with a graduated cylinder

Three 100 μL aliquots are retained from each preservation mixture in a 96-well plate in order to determine the colony forming units (CFUs) of the culture. For CFU determination, each aliquot is serially diluted 10-fold in PBS and 5 μL of each dilution was spotted onto plates to determine CFUs.

For preservation, 100 μL of each preservation mixture was dispensed into a 2 mL serum vial, which was then sealed with a lyophilization cap and placed the vials in an aluminum lyophilizer block. The vials were frozen at −80° C. for at least one hour and then the vials were transferred to the lyophilizer in the aluminum block. Lypholization caps were changed to the open position and the following lyophilization program was executed:
(a) Freeze at −17° C. at atmospheric pressure for 30 minutes
(b) Freeze at −17° C. at 1000 mTorr for 15 minutes
(c) Freeze at −17° C. at 300 mTorr for 15 minutes
(d) Incubate at 30° C. at 300 mTorr for 24 hours
(e) Incubate at 40° C. at 300 mTorr for 24 hours
(f) Hold at 25° C.

Alternative lypholization protocols may also be used such as freezing at a temperature between −20° C. and 0° C. at a vacuum pressure less than 1000 mTorr (e.g., 900 mTorr, 800 mTorr, 700 mTorr, etc.). Primary drying steps can include incubation at a temperature between 10° C. and 30° C. at a given vacuum pressure level. Secondary drying steps can include incubation at a temperature that is greater than the temperature used during primary drying at the same vacuum level.

All vials are then removed from the lyophilizer and rehydrated in the following manner:
(a) 1 mL of sterile PBS is added to each vial (effectively a 10× dilution to the initial preservation mixture) and reconstituted by slowly pipetting up and down. This mixture is then diluted 6 additional logs (for a total dilution of E-07) and a 5 μL aliquot from each vial is spot plated for CFU determination.
(b) A separate aliquot of the reconstituted PBV product is streaked onto a plate as the starting plate (a “rescue” plate) for re-inoculation in subsequent.

A second and third round of PBV is then performed according to the protocol described above, using the “rescue” plates as the initial source of bacteria for inoculation of the seed tube.

### Example 2: Serial Preservation Challenges of Ruminococcus bovis

R. bovis (ASCUSDY10) was subjected to a series of preservation challenges and recoveries in order to improve yield through a serial preservation process. R. bovis was subjected to three rounds of Preservation by Vaporization (PBV) challenges according to the protocol described in Example 1. The results from Round 1-3 for ASCUSDY10 are presented in Table 4 below. As shown, there was a dramatic increase in the Survival % of Colony Forming Units (CFU)/mL for DY10 from Round 1 (RCB) to Round 2 (Rescue 1).

TABLE 4
CFU Titer and PBV survival of R. bovis

| Round | Microbe | Inoculant source | Titer (CFU/mL) | PBV Survival (%) |
|---|---|---|---|---|
| 1 | ASCUSDY10 | RCB | 7.70E+08 | 0.0013% |
| 2 | ASCUSDY10 | Rescue plate Round 1 | 6.70E+08 | 30% |
| 3 | ASCUSDY10 | Rescue plate Round 2 | 4.93E+08 | 20% |

The genomes of the RCB isolate and the Round 3 isolate of ASCUSDY10 were sequenced to determine any genomic changes as a result of the serial passage. Briefly, DNA was isolated from R. bovis using a Qiagen Powersoil Pro kit. Short read sequencing libraries were prepared from the isolated DNA using the Nextera XT kit (Illumina, San Diego, Calif.) by the manufacturer's recommended protocol. Libraries were sequenced on an Illumina MiSeq (1×300 bp). Reads were mapped to the reference genome using bowtie2 (Langmead B, Salzberg S. (2012) Fast gapped-read alignment with Bowtie 2. Nature Methods. 9: 357-359) and analyzed for mutations using breseq (Deatherage D E, Barrick J E. (2014) Identification of mutations in laboratory-evolved microbes from next-generation sequencing data using breseq. Methods Mol. Biol. 1151: 165-188).

A summary of the mutations is presented in Table 5 below. Mutations 7 and 8 are silent mutations and unlikely to result in significant effects. Mutations 2, 3, 5, and 6 affect either integrases or transposases and are unlikely to affect preservation tolerance. Mutation 1 is likely the key mutation resulting in the improvement of preservation tolerance in ASCUSDY10. It occurs 4 bp upstream of the Galactose operon repressor, GalR-LacI. This key protein represses transcription of a host of genes related to carbohydrate uptake and metabolism. As cryoprotectant uptake, often in the form of non-reducing sugars, is a key step in preservation tolerance, a change in the regulation of sugar uptake could result in a dramatic improvement in preservation tolerance. The phosphomannomutase could provide another key mutation, perhaps disrupting the metabolism of preservation sugars and enabling intracellular accumulation.

TABLE 5
R. bovis mutation summary

| | Mutation | | | |
|---|---|---|---|---|
| | position | Change | Description | Protein Description |
| 1 | 676,590 | G→T | intergenic (−223/−4) | Melibiose carrier protein, Na+, |
| | | | | melibiose symporter/Galactose |
| | | | | operon repressor, GalR-LacI family |
| | | | | of transcriptional regulators |
| 2 | 759,729 | 2 bp→TA | coding (306-307/633 nt) | hypothetical protein (integrase) |
| 3 | 759,735 | T→G | K101Q (AAG→CAG) | hypothetical protein (integrase) |
| 4 | 1,403,355 | (A)_{5→4} | coding (23/1503 nt) | Phosphomannomutase |
| 5 | 1,450,594 | 3 bp→TTC | coding (55-57/300 nt) | hypothetical protein (transposase) |
| 6 | 1,546,754 | +T | coding (84/126 nt) | hypothetical protein (transposase) |
| 7 | 1,667,526 | C→T | Y399Y (TAC→TAT) | hypothetical protein |
| 8 | 2,124,083 | C→A | G414G (GGC→GGA) | Excinuclease ABC subunit B |
| 9 | 2,437,094 | +AC | coding (233/240 nt) | hypothetical protein |
| | | | | (stage II sporulation protein) |

### Example 3: Serial Preservation Challenges of Succinivibrio dextrinosolvens

S. dextrinosolvens (ASCUSBF53) was subjected to the PBV challenge described in example 1. The results from Round 1-3 for ASCUSBF53 are presented below in Table 6. As shown, there was an increase in both the PBV Survival % and the maximum culture titer achieved from the initial culture through the preservation challenge.

TABLE 6
CFU Titer and PBV survival of S. dextrinosolvens

| | | | Titer | PBV Survival |
|---|---|---|---|---|
| Round | Microbe | Inoculant source | (CFU/mL) | (%) |
| 1 | ASCUSBF53 | RBC | 6.53E+08 | 3% |
| 2 | ASCUSBF53 | Rescue plate Round 1 | 1.11E+09 | 14% |
| 3 | ASCUSBF53 | Rescue plate Round 2 | 2.20E+09 | 15% |

### Example 4: Cryopreservation of Caecomyces spp.

Caecomyces spp. (ASCUSDY30) was subjected to a series of cryopreservation challenges and recoveries in order to select for a population more resistant to cryostorage at −80° C. Caecomyces spp. ASCUSDY30 was grown in a modified version of Medium C without rumen fluid and 1% (w/v) glucose (Solomon et al., (2016) Early-branching gut fungi possess a large, comprehensive array of biomass-degrading enzymes. Science. 351: 1192-1195). Cultures were grown for 72 hours prior to harvest by centrifugation at 4,000×g for 10 min at 4° C. Cultures were resuspended in an anaerobic preservation solution consisting of 5% Sorbitol and 15% Sucrose prior to freezing at −80° C. Frozen cultures were assessed for survival through a TFU enumeration method using roll tubes as previously described for anaerobic fungi (Joblin K. (1981) Isolation, Enumeration, and Maintenance of Rumen Anaerobic Fungi in Roll Tubes. Applied and Environmental Microbiology. 42:1119-1122).

As shown in Table 7, the initial population had a survival of Thallus Forming Units (TFU)/mL lower than the limit of detection for the assay. After recovering from this population and challenging again, the TFU/mL of the surviving population was at least 10 times higher than in Round 1.

TABLE 7
Post-freeze survival of Caecomyces spp.

| | | | Post-freeze survival |
|---|---|---|---|
| Round | Microbe | Inoculant source | (TFU/mL) |
| 1 | ASCUSDY30 | RBC | <20 |
| 2 | ASCUSDY30 | Rescue plate Round 1 | 220 |

1. A method of improving microbe viability after preservation comprising:
a. subjecting a population of target microbial cells to a first preservation challenge to provide a population of challenged microbial cells;
b. harvesting viable challenged microbial cells from the population of challenged microbial cells;
c. preserving the viable challenged microbial cells to provide a population of preserved viability-enhanced microbial cells; and
d. preparing a product using the population of preserved viability-enhanced microbial cells.

2. The method of claim 1, wherein the first preservation challenge includes one of freeze drying, lyophilization, cryopreservation, preservation by evaporation, preservation by foam formation, vitrification, stabilization by glass formation, preservation by vaporization, spray drying, adsorptive drying, extrusion, or fluid bed drying.

3. The method of claim 1, wherein preserving the viable challenged cells includes freeze drying, lyophilization, cryopreservation, preservation by evaporation, preservation by foam formation, vitrification, stabilization by glass formation, preservation by vaporization, spray drying, adsorptive drying, extrusion drying, or fluid bed drying.

4. The method of claim 1, further comprising subjecting the population of challenged cells to at least one additional preservation challenge

5. A method for microbe viability enhancement and preservation, the method comprising:
a. subjecting a population of target microbial cells to a first preservation challenge to provide a first population of challenged microbial cells;
b. harvesting viable challenged microbial cells from the first population of challenged microbial cells to provide a first population of viable challenged microbial cells;
c. subjecting the first population of viable challenged microbial cells to a second preservation challenge to provide a second population of challenged microbial cells;
d. harvesting viable challenged microbial cells from the second population of challenged microbial cells to provide a second population of viable challenged microbial cells;
e. preserving the second population of viable challenged microbial cells to provide a population of preserved viability-enhanced microbial cells; and
f. preparing a product using the population of preserved viability-enhanced microbial cells.

6. The method of claim 5, wherein the first preservation challenge and the second preservation challenge are of the same challenge type.

7. The method of claim 5, wherein the first preservation challenge and the second preservation challenge are of different challenge types.

8. The method of claim 5, wherein the first preservation challenge and the second preservation challenge are selected from a combination described in Table 1.

9. The method of claim 5, further comprising subjecting the second population of challenged cells to at least one additional preservation challenge.

10. The method of claim 5, wherein preserving the second viable challenged cell population includes freeze drying, lyophilization, cryopreservation, preservation by evaporation, preservation by foam formation, vitrification, stabilization by glass formation, preservation by vaporization, spray drying, adsorptive drying, extrusion drying, or fluid bed drying.

11. The method of claim 1, wherein the population of target microbial cells comprises a Clostridium spp. bacterium, a Succinivibrio spp. bacterium, a Butyrivibio spp. bacterium, a Bacillus spp. bacterium, a Lactobacillus spp. bacterium, a Prevotella spp. bacterium, a Syntrophococcus spp. bacterium, a Ruminococcus spp. bacterium, a Caecomyces spp. fungus, a Pichia spp. fungus, an Orpinomyces spp. fungus, a Piromyces spp. fungus, or a species of the Lachnospiraceae family.

12. (canceled)

13. (canceled)

14. The method of claim 11, wherein:
a. the Clostridium spp. comprises a 16S rRNA sequence comprising at least 97% sequence identity to SEQ ID NO: 1, SEQ ID NO: 3, SEQ ID NO: 5, or SEQ ID NO: 6;
b. the Succinivibrio spp. comprises a 16S rRNA sequence comprising at least 97% sequence identity to SEQ ID NO: 11;
c. the Pichia spp. comprises an ITS sequence comprising at least 97% sequence identity to SEQ ID NO: 2;
d. the Bacillus spp. comprises a 16S rRNA sequence comprising at least 97% sequence identity to SEQ ID NO: 4;
e. the Lactobacillus spp. comprises a 16S rRNA sequence comprising at least 97% sequence identity to SEQ ID NO: 7, SEQ ID NO: 8, or SEQ ID NO: 9;
f. the Prevotella spp. comprises a 16S rRNA sequence comprising at least 97% sequence identity to SEQ ID NO: 10; or
g. the species of the Lachnospiraceae family comprises a 16S rRNA sequence comprising at least 97% sequence identity to SEQ ID NO: 12.

15. The method of claim 1, wherein the population of target microbial cells comprises a Ruminococcus bovis bacterium, a Succinivibrio dextrinosolvens bacterium, or a Caecomyces spp. fungus.

16. The method of claim 1, wherein the population of target microbial cells comprises a Clostridium butyricum bacterium, a Pichia kudriazevii fungus, a Butyrivibio fibrosolvens bacterium, a Ruminococcus bovis bacterium, or a Succinivibrio dextrinosolvens bacterium.

17. A product prepared by the methods of claim 1, comprising a population of preserved viability-enhanced microbial cells.

18. The product of claim 17, wherein the population of preserved viability-enhanced microbial cells comprises a Clostridium spp. bacterium, a Succinivibrio spp. bacterium, a Caecomyces spp. bacterium, a Pichia spp. fungus, a Butyrivibio spp. bacterium, an Orpinomyces spp. fungus, a Piromyces spp. fungus, a Bacillus spp. bacterium, a Lactobacillus spp. bacterium, a Prevotella spp. bacterium, a Syntrophococcus spp. bacterium, a Ruminococcus spp bacterium, or a species of the Lachnospiraceae family.

19. The product of claim 18, wherein:
a. the Clostridium spp. comprises a 16S rRNA sequence comprising at least 97% sequence identity to SEQ ID NO: 1, SEQ ID NO: 3, SEQ ID NO: 5, or SEQ ID NO: 6;
b. the Succinivibrio spp. comprises a 16S rRNA sequence comprising at least 97% sequence identity to SEQ ID NO: 11;
c. the Pichia spp. comprises an ITS sequence comprising at least 97% sequence identity to SEQ ID NO: 2;
d. the Bacillus spp. comprises a 16S rRNA sequence comprising at least 97% sequence identity to SEQ ID NO: 4;
e. the Lactobacillus spp. comprises a 16S rRNA sequence comprising at least 97% sequence identity to SEQ ID NO: 7, SEQ ID NO: 8, or SEQ ID NO: 9; or
f. the Prevotella spp. comprises a 16S rRNA sequence comprising at least 97% sequence identity to SEQ ID NO: 10; or
g. the species of the Lachnospiraceae family comprises a 16S rRNA sequence comprising at least 97% sequence identity to SEQ ID NO: 12.


SEQUENCE LISTING (12 sequences)

SEQ ID NO: 1 - DNA, 225 bp, Unknown
  Encodes 16S rRNA from Clostridium sensu stricto, Ascusb_3138 DY20
agagtttgatcctggctcaggacgaacgctggcggcgtgcttaacacatgcaagtcgagcgatgaagttccttcgggaatggattagcggcggacgggtgagtaacacgtgggtaacctgcctcatagaggggaatagcctttcgaaaggaagattaataccgcataagattgtagcaccgcatggtgcagcaattaaaggagtaatccgctatgagatggaccc

SEQ ID NO: 2 - DNA, 225 bp, Unknown
  ITS2 sequence of Candida xylopsoci, Ascusf_15, DY21
tcctccgcttattgatatgcttaagttcagcgggtattcctacctgatttgaggtcgagctttttgttgtctcgcaacactcgctctcggccgccaagcgtccctgaaaaaaagtctagttcgctcggccagcttcgctccctttcaggcgagtcgcagctccgacgctctttacacgtcgtccgctccgctcccccaactctgcgcacgcgcaagatggaaacg

SEQ ID NO: 3 - DNA, 225 bp, Unknown
  Encodes 16S rRNA from Clostridium IV, Ascusb_5 DY10
agagtttgatcctggctcaggatgaacgctggcggcgtgcctaacacatgcaagtcgaacggaacttctttgacagaattcttcggaaggaagttgattaagtttagtggcggacgggtgagtaacgcgtgagtaacctgcctttgagaggggaataacttcccgaaagggatgctaataccgcataaagcatagaagtcgcatggcttttatgccaaagattta

SEQ ID NO: 4 - DNA, 224 bp, Unknown
  Encodes 16S rRNA from Bacillus, Ascusbbr_33(A) CR11
agatttgatcatggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttaca

SEQ ID NO: 5 - DNA, 224 bp, Unknown
  Encodes 16S rRNA sequence from Clostridium, Ascusbbr_105932 BR21
agagtttgatcctggctcaggatgaacgctggcggcgtgcttaacacatgcaagtcgagcgaagcagttttaaggaagttttcggatggaattaaaattgacttagcggcggacgggtgagtaacgcgtgggtaacctgcctcatacagggggataacagttagaaatgactgctaataccgcataagcgcacagtgctgcatagcacagtgtgaaaaactccg

SEQ ID NO: 6 - DNA, 224 bp, Unknown
  Encodes 16S rRNA sequence from Clostridium, Ascusbbr_2676 BR67
agagtttgatcatggctcaggacgaacgctggcggcgtgcttaacacatgcaagtcgagcgatgaagttccttcgggaacggattagcggcggacgggtgagtaacacgtgggtaacctgcctcatagaggggaatagccttccgaaaggaagattaataccgcataagattgtagtttcgcatgaaacagcaattaaaggagtaatccgctatgagatggacc

SEQ ID NO: 7 - DNA, 224 bp, Unknown
  Encodes 16S rRNA from Lactobacillus, Ascusbbr_5796(A) BR16
agatttgctcctggctcaggacgaacgctggcggcgtgcctaatacatgcaagtcgagcgagcggaactaacagatttacttcggtaatgacgttaggaaagcgagcggcggatgggtgagtaacacgtggggaacctgccccatagtctgggataccacttggaaacaggtgctaataccggataagaaagcagatcgcatgatcagcttttaaaaggcggcg

SEQ ID NO: 8 - DNA, 224 bp, Unknown
  Encodes 16S rRNA from Lactobacillus, Ascusbbr_5796(B) BR16
agagtttgatcatggctcaggacgaacgctggcggcgtgcctaatacatgcaagtcgagcgagcggaactaacagatttacttcggtaatgacgttaggaaagcgagcggcggatgggtgagtaacacgtggggaacctgccccatagtctgggataccacttggaaacaggtgctaataccggataagaaagcagatcgcatgatcagcttttaaaaggcggc

SEQ ID NO: 9 - DNA, 225 bp, Unknown
  Encodes 16S rRNA from Lactobacillus, Ascusbbr_5796(C) BR16
agagtttgatcctggctcaggacgaacgctggcggcgtgcctaatacatgcaagtcgagcgagcggaactaacagatttacttcggtaatgacgttaggaaagcgagcggcggatgggtgagtaacacgtggggaacctgccccatagtctgggataccacttggaaacaggtgctaataccggataagaaagcagatcgcatgatcagcttttaaaaggcggcg

SEQ ID NO: 10 - DNA, 225 bp, Unknown
  Encodes 16S rRNA from Prevotella, Ascusbbf_4 BY41
agagtttgatcctggctcaggatgaacgctagctacaggcttaacacatgcaagtcgaggggaaacgacatagagtgcttgcactttatgggcgtcgaccggcgaatgggtgagtaacgcgtatccaacctgcccttgaccgagggatagcccagtgaaaactgaattaatacctcatgttctcctcagacggcatcagacgaggagcaaagattaatcggtcaa

SEQ ID NO: 11 - DNA, 225 bp, Unknown
  Encodes 16S rRNA from Succinivibrio, Ascusbbf_154 BF53
agagtttgatcatggctcagattgaacgctggcggcaggcctaatacatgcaagtcgaacggtaacataggaaaagcttgcttttcctgatgacgagtggcggacgggtgagtaaagtttgggaagctacctgatagagggggacaacagttggaaacgactgctaataccgcatacagcctgagggtgaaagcagcaatgcgctatcagatgcgcccaaatggg

SEQ ID NO: 12 - DNA, 225 bp, Unknown
  Encodes 16S rRNA from Lachnospiraceae, Ascusbbf_876 BF65
agagtttgatcctggctcaggatgaacgctggcggcgtgcctaacacatgcaagtcgagcggagtgaagagagcttgcttttttcacttagcggcggatgggtgaggaacgcgtggggaacctgcctctcacagggggataacagctggaaacggctgttaataccgcatatgcacacagtgccgcatggcacagggtggaaagaaattcggtgagagatggacc